(19) United States
(12) Patent Application Publication
(10) Pub. No.: US 2024/0116914 A1
(43) Pub. Date: Apr. 11, 2024

(54) COMPOUND AND ORGANIC ELECTROLUMINESCENT ELEMENT USING SAID COMPOUND

(71) Applicant: HODOGAYA CHEMICAL CO., LTD., Tokyo (JP)

(72) Inventors: Eriko CHIBA, Tokyo (JP); Takeshi YAMAMOTO, Tokyo (JP); Kouki KASE, Tokyo (JP); Yuta HIRAYAMA, Tokyo (JP); Shuichi HAYASHI, Tokyo (JP)

(73) Assignee: HODOGAYA CHEMICAL CO., LTD., Tokyo (JP)

(21) Appl. No.: 18/273,421

(22) PCT Filed: Feb. 10, 2022
(86) PCT No.: PCT/JP2022/005500

§ 371 Date: Jul. 20, 2023

(30) Foreign Application Priority Data
Feb. 15, 2021 (JP) 2021-021890

(51) Int. Cl. C07D 413/14; C07C 15/24; C07D 209/90; C07D 239/26; C07D 307/91; C07D 401/10; C07D 401/14; C07D 417/14; H10K 85/20; H10K 85/60

(52) U.S. Cl. CPC C07D 413/14; C07C 15/24; C07D 209/90; C07D 239/26; C07D 307/91; C07D 401/10; C07D 401/14; C07D 417/14; H10K 85/20; H10K 85/615; H10K 85/654; H10K 85/6572; H10K 85/6574; H10K 85/6576

(57) ABSTRACT

It is an object of the present invention to provide a compound for a capping layer that has a high refractive index and a low extinction coefficient in a range of 450 nm to 750 nm to improve the light extraction efficiency of an organic EL element. The present invention has focused on the fact that compounds having a benzene skeleton at the center thereof are excellent in stability and also durability in the form of a thin film, and that the refractive index thereof can be improved by modifying the molecular structure, and thus, molecules have been designed. Provided is a compound represented by a general formula (1), and an organic EL element with excellent luminance efficiency is obtained by using that compound as a constituent material of a capping layer.

### TECHNICAL FIELD

The present invention relates to a compound suitable for a self-emissive electronic element favorably used in various display devices, and particularly a compound suitable for an organic electroluminescent element (hereinafter abbreviated as an “organic EL element”), and also relates to an organic EL element including the compound.

### BACKGROUND ART

Since organic EL elements are self-emissive elements, they have larger brightness and better viewability than elements including liquid crystals, and can thus provide a clearer display. For these reasons, active studies have been carried out on organic EL elements.

In 1987, C. W. Tang et al. of Eastman Kodak Company developed an element having a layered structure in which various functions were assigned to different materials, and thus made a practical organic EL element including organic materials. They made an organic EL element having a layered structure including a layer of a fluorescent substance capable of transporting electrons and a layer of an organic substance capable of transporting holes, and injected both charges into the layer of the fluorescent substance to thereby cause the layer to emit light, and the organic EL element thus achieved a luminance as high as 1,000 cd/m² or more at a voltage of 10 V or less (see Patent Literatures 1 and 2, for example).

Organic EL elements have been heretofore much improved to put them to practical use. Electroluminescent elements with a bottom emission structure, which emit light from the bottom thereof, have been suggested in which an anode, a hole-injecting layer, a hole-transporting layer, a light-emitting layer, an electron-transporting layer, an electron-injecting layer, and a cathode are sequentially provided on a substrate to subdivide various functions in the multi-layered structure even further, and such electroluminescent elements successfully have high efficiency and durability (see Non-Patent Literature 1, for example).

Recently, light-emitting elements with a top emission structure are coming into use. In the light-emitting element with a top emission structure, a metal having a high work function is used as an anode, and light is extracted from the top side of the element.

A light-emitting element with a bottom emission structure results in a limited area of the light-emitting portion, since light is extracted from its bottom side, which has circuits for pixels. In contrast, the light-emitting element with a top emission structure advantageously enables a large light-emitting area since light extracted from the top side is not blocked by the circuits for pixels. In light-emitting elements with a top emission structure, translucent electrodes made of LiF/Al/Ag (see Non-Patent Literature 2, for example), Ca/Mg (see Non-Patent Literature 3, for example), LiF/MgAg, or the like are used for a cathode.

In such light-emitting elements, when light is emitted by a light-emitting layer and incident on another film, the light is totally reflected at an interface between the light-emitting layer and the other film, if the light is incident at a certain angle or larger.

Accordingly, it is only a part of the emitted light that is available. Recently, light-emitting elements have been proposed in which a “capping layer” with a high refractive index is provided on the outside of a translucent electrode with a low refractive index, in order to improve the light extraction efficiency (see Non-Patent Literatures 2 and 3, for example).

The effect of the capping layer in a light-emitting element with a top emission structure including Ir(ppy)₃ as a light-emitting material was as follows: while a light-emitting element without a capping layer has a current efficiency of 38 cd/A, a light-emitting element including a ZnSe film with a thickness of 60 nm as a capping layer has a current efficiency of 64 cd/A, which indicates that the efficiency is improved about 1.7 times. Furthermore, it has been shown that the maximum point of the transmittance of the translucent electrode and the capping layer and the maximum point of the efficiency do not absolutely match each other, and it is thus indicated that the maximum point of the light extraction efficiency depends on the interference effects (see Non-Patent Literature 3, for example).

Conventionally, it has been proposed to use a fine metal mask to form a capping layer, but the metal mask is disadvantageously deformed by heat when used at a high temperature, which deteriorates the positioning precision. For example, ZnSe has a high melting point of 1100° C. or higher (see Non-Patent Literature 3, for example), and thus cannot be vapor-deposited at an accurate position with a fine metal mask, which may affect the light-emitting element itself. Furthermore, film formation by sputtering also affects the light-emitting element. Thus a capping layer made of an inorganic material as a constituent material cannot be suitably used.

In addition, it has been proposed to use tris(8-hydroxyquinoline)aluminum (hereinafter abbreviated as “Alq₃”) for a capping layer for adjusting the refractive index (see Non-Patent Literature 2, for example). Alq₃ is known as an organic EL material commonly used as a green light-emitting material or electron-transporting material, and exhibits small absorption at about 450 nm, and thus, when it is used for blue light-emitting elements, problems of a poor color purity and a small efficiency in light extraction arise.

In light of improving the characteristics of an organic EL element and also significantly improving the light extraction efficiency thereof, there is a demand for a material for a capping layer that has a high refractive index and a low extinction coefficient and also has excellent stability and durability in the form of a thin film.

### CITATION LIST

### Patent Literature

Patent Literature 1: U.S. Pat. No. 5,792,557
Patent Literature 2: U.S. Pat. No. 5,639,914
Patent Literature 3: EP 2684932
Patent Literature 4: US 20140225100

### Non-Patent Literature

Non-Patent Literature 1: Proceedings of the 9th Meeting of the Japan Society of Applied Physics, pp. 55-61 (2001)
Non-Patent Literature 2: Appl. Phys. Let., 78, 544 (2001)
Non-Patent Literature 3: Appl. Phys. Let., 82, 466 (2003)
Non-Patent Literature 4: Appl. Phys. Let., 98, 083302 (2011)

### SUMMARY OF INVENTION

It is an object of the present invention to provide a compound that has a high refractive index and a low extinction coefficient in a wavelength range of 450 to 750 nm and is suitable for use in a capping layer of an organic EL element. It is another object of the present invention to provide an organic EL element including such a compound to improve the light extraction efficiency of the element.

A compound suitably used for a capping layer of an organic EL element has the following physical properties, for example: (1) a high refractive index, (2) a low extinction coefficient, (3) vapor-depositability, (4) good stability in the form of a thin film, and (5) a high glass transition point. The present invention intends to provide an organic EL element having the following physical properties, for example: (1) high light extraction efficiency, (2) no deterioration in the color purity, (3) the ability to transmit light without change over time, and (4) a long life.

To achieve the above-described objects, the inventors have focused on the fact that compounds having a benzene skeleton at the center thereof are excellent in stability and also durability in the form of a thin film, and that the refractive index thereof can be improved by modifying the molecular structure. The inventers have thus designed molecules, produced organic EL elements including the compounds as a constituent material for the capping layer, and thoroughly evaluated the properties of the elements, and as a result, the present invention has been accomplished.

Specifically, the present invention is directed to a compound represented by a general formula (1) below, and an organic EL element including the compound, and in more detail, the present invention directed to the following.

1) A compound represented by a general formula (1) below.

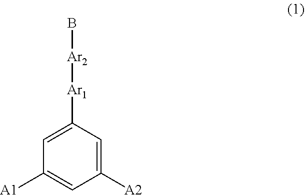

In the formula (1), B represents a substituted or unsubstituted aromatic hydrocarbon group, a substituted or unsubstituted aromatic heterocyclic group, a substituted or unsubstituted fused polycyclic aromatic group, or a substituted or unsubstituted aryloxy group. Ar₁ and Ar₂ may be the same or different, and represent a substituted or unsubstituted divalent aromatic hydrocarbon group, a substituted or unsubstituted divalent aromatic heterocyclic group, a substituted or unsubstituted divalent fused polycyclic aromatic group, or a single bond. A1 and A2 may be the same or different, and represent a monovalent group represented by a general formula (2) below.

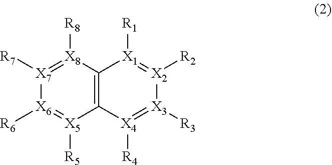

In the formula (2), R₁ to R₈ may be the same or different, and represent a binding site, a hydrogen atom, a deuterium atom, a fluorine atom, a chlorine atom, a cyano group, a nitro group, a linear or branched alkyl group having 1 to 6 carbon atoms and optionally having a substituent, a cycloalkyl group having 5 to 10 carbon atoms and optionally having a substituent, a linear or branched alkenyl group having 2 to 6 carbon atoms and optionally having a substituent, a linear or branched alkyloxy group having 1 to 6 carbon atoms and optionally having a substituent, a cycloalkyloxy group having 5 to 10 carbon atoms and optionally having a substituent, a substituted or unsubstituted aryloxy group, a substituted or unsubstituted aromatic hydrocarbon group, a substituted or unsubstituted aromatic heterocyclic group, or a substituted or unsubstituted fused polycyclic aromatic group, and any one of R₁ to R₈ represents a binding site. X₁ to X₈ may be the same or different, and represent a nitrogen atom or a carbon atom. The number of X₁ to X₈ representing a nitrogen atom is zero to two, and the nitrogen atom does not have a corresponding group of R₁ to R₈ bonded thereto.

2) The compound as set forth in 1), wherein A1 and A2 are a monovalent group represented by a general formula (3a), (3b), or (3c) below.

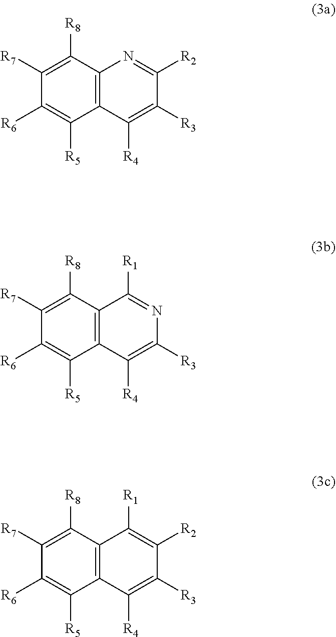

R₁ to R₈ in the formulas (3a), (3b), or (3c) are the same as defined in the formula (2).

3) The compound as set forth in 1) or 2), wherein B is a substituted or unsubstituted naphthalenyl group, phenanthrenyl group, dibenzofuranyl group, dibenzothiophenyl group, fluorenyl group, carbazolyl group, benzofuranyl group, or benzothiophenyl group.

4) An organic thin film containing the compound as set forth in any one of 1) to 3), having a refractive index of 1.70 or more in a wavelength range of 450 nm to 750 nm.

5) An organic electroluminescent element including, at least, an anode, a hole-transporting layer, a light-emitting layer, an electron-transporting layer, a cathode, and a capping layer arranged, in this order, wherein the capping layer is the organic thin film as set forth in 4).

6) An electronic element including a pair of electrodes and at least one organic layer interposed therebetween, wherein the organic layer contains the compound as set forth in any one of 1) to 3).

7) An electronic device including the electronic element as set forth in 6).

In the present invention, the term “unsubstituted” in the expression “substituted or unsubstituted” means that no hydrogen atom is replaced with a substituent.

In the present invention, the term “hydrogen atom” encompasses isotopes with different neutron numbers, that is, light hydrogen and heavy hydrogen.

In the present invention, the “substituent” in the expression “substituted or unsubstituted” may specifically refer to a cyano group, a nitro group, a halogen atom, a substituted or unsubstituted alkyl group having 1 to 3 carbon atoms, a substituted or unsubstituted phenyl group, or a substituted or unsubstituted alkoxy group having 1 to 3 carbon atoms.

The “aromatic hydrocarbon group”, the “aromatic heterocyclic group”, or the “fused polycyclic aromatic group” in the “substituted or unsubstituted aromatic hydrocarbon group”, the “substituted or unsubstituted aromatic heterocyclic group”, or the “substituted or unsubstituted fused polycyclic aromatic group” represented by B and Ar₁ to Ar₂ in the general formula (1) and R₁ to R₈ in the general formula (2) may specifically refer to a group selected from aryl groups having 6 to 30 carbon atoms and heteroaryl groups having 2 to 20 carbon atoms, such as a phenyl group, a biphenylyl group, a terphenylyl group, a naphthyl group, an anthracenyl group, a phenanthrenyl group, a fluorenyl group, a spirobifluorenyl group, an indenyl group, a pyrenyl group, a perylenyl group, a fluoranthenyl group, a triphenylenyl group, a pyridyl group, a pyrimidinyl group, a triazinyl group, a furyl group, a pyrrolyl group, a thienyl group, a quinolyl group, an isoquinolyl group, a benzofuranyl group, a benzothienyl group, an indolyl group, a carbazolyl group, a benzooxazolyl group, a benzothiazolyl group, a quinoxalinyl group, a benzimidazolyl group, a pyrazolyl group, a dibenzofuranyl group, a dibenzothienyl group, a naphthyridinyl group, a phenanthrolinyl group, an acridinyl group, and a carbolinyl group.

The “linear or branched alkyl group having 1 to 6 carbon atoms”, the “cycloalkyl group having 5 to 10 carbon atoms”, the “linear or branched alkenyl group having 2 to 6 carbon atoms”, the “linear or branched alkyloxy group having 1 to 6 carbon atoms”, the “cycloalkyloxy group having 5 to 10 carbon atoms”, or the “aryloxy group” in the “linear or branched alkyl group having 1 to 6 carbon atoms and optionally having a substituent”, the “cycloalkyl group having 5 to 10 carbon atoms and optionally having a substituent”, the “linear or branched alkenyl group having 2 to 6 carbon atoms and optionally having a substituent”, the “linear or branched alkyloxy group having 1 to 6 carbon atoms and optionally having a substituent”, the “cycloalkyloxy group having 5 to 10 carbon atoms and optionally having a substituent”, or the “substituted or unsubstituted aryloxy group” represented by R₁ to R₈ in the general formula (2) may specifically refer to a methyl group, an ethyl group, an n-propyl group, an isopropyl group, an n-butyl group, an isobutyl group, a tert-butyl group, an n-pentyl group, an isopentyl group, a neopentyl group, an n-hexyl group, a cyclopentyl group, a cyclohexyl group, a 1-adamanthyl group, a 2-adamanthyl group, a vinyl group, an aryl group, an isopropenyl group, a 2-butenyl group, a methyloxy group, an ethyloxy group, an n-propyloxy group, a cyclopentyloxy group, a cyclohexyloxy group, a 1-adamantyloxy group, a phenyloxy group, a tolyloxy group, or a biphenyloxy group.

The “substituent” in the “substituted aromatic hydrocarbon group”, the “substituted aromatic heterocyclic group”, the “substituted fused polycyclic aromatic group”, the “linear or branched alkyl group having 1 to 6 carbon atoms and optionally having a substituent”, the “cycloalkyl group having 5 to 10 carbon atoms and optionally having a substituent”, or the “linear or branched alkenyl group having 2 to 6 carbon atoms and optionally having a substituent” represented by B and Ar₁ to Ar₂ in the general formula (1) and R₁ to R₈ in the general formula (2) may specifically refer to: a heavy hydrogen atom, a cyano group, or a nitro group; a halogen atom such as a fluorine atom, a chlorine atom, a bromine atom, or an iodine atom; a silyl group such as a trimethylsilyl group or a triphenylsilyl group; a linear or branched alkyl group having 1 to 6 carbon atoms such as a methyl group, an ethyl group, or a propyl group; a linear or branched alkyloxy group having 1 to 6 carbon atoms such as a methyloxy group, an ethyloxy group, or a propyloxy group; an alkenyl group such as a vinyl group or an aryl group; an aryloxy group such as a phenyloxy group or a tolyloxy group; an arylalkyloxy group such as a benzyloxy group or a phenethyloxy group; an aromatic hydrocarbon group or a fused polycyclic aromatic group such as a phenyl group, a biphenylyl group, a terphenylyl group, a naphthyl group, an anthracenyl group, a phenanthrenyl group, a fluorenyl group, a spirobifluorenyl group, an indenyl group, a pyrenyl group, a perylenyl group, a fluoranthenyl group, or a triphenylenyl group; or an aryl group having 6 to 30 carbon atoms or a heteroaryl group having 2 to 20 carbon atoms such as a pyridyl group, a thienyl group, a furyl group, a pyrrolyl group, a quinolyl group, an isoquinolyl group, a benzofuranyl group, a benzothienyl group, an indolyl group, a carbazolyl group, a benzooxazolyl group, a benzothiazolyl group, a quinoxalinyl group, a benzimidazolyl group, a pyrazolyl group, a dibenzofuranyl group, a dibenzothienyl group, or a carbolinyl group. These substituents may be further substituted with any of the above-listed substituents. Furthermore, a substituent and the benzene ring substituted therewith, or adjacent substituents of a plurality of substituents on the same benzene ring may be bonded to each other via a single bond, a substituted or unsubstituted methylene group, an oxygen atom, or a sulfur atom to form a ring.

A1 and A2 in the general formula (1) are each a monovalent group represented by the general formula (2) above and have any one of R₁ to R₈ as a binding site. The group represented by the general formula (2) above is preferably a monovalent group represented by the general formula (3a), (3b), or (3c) above, and more preferably a group represented by the general formula (3a) or (3c) above. A1 and A2 are preferably the same.

When A1 and A2 in the general formula (1) are a group represented by the general formula (3a) above, the binding site is preferably R₂, R₃, R₈, or R₆, in view of the refractive index and the thermal resistance. Among R₂ to R₈, those that are not the binding site are preferably all hydrogen atoms, in view of ease of synthesis.

When A1 and A2 in the general formula (1) are a group represented by the general formula (3c) above, the binding site is preferably R₂ in view of the refractive index and the thermal resistance. Among R₁ to R₈, those that are not the binding site are preferably all hydrogen atoms, in view of ease of synthesis.

Ar₁ and Ar₂ in the general formula (1) are preferably each independently selected from a phenylene group, a pyridylene group, a pyrimidinylene group, and a single bond, in view of the refractive index and the thermal resistance.

B in the general formula (1) is more preferably a substituted or unsubstituted naphthalenyl group, phenanthrenyl group, dibenzofuranyl group, dibenzothiophenyl group, fluorenyl group, carbazolyl group, benzofuranyl group, or benzothiophenyl group.

The compound represented by the general formula (1) above of the present invention has a refractive index of preferably 1.70 or more, and more preferably 1.85 or more, in a wavelength range of 450 to 700 nm.

Since the compound represented by the general formula (1) above of the present invention has a high refractive index and a low extinction coefficient in a wavelength range of 450 to 750 nm, it is possible to obtain an organic EL element having significantly improved efficiency in light extraction, when this compound is used in a capping layer provided outside the transparent or translucent electrode of the organic EL element and having a higher refractive index than the translucent electrode.

### BRIEF DESCRIPTION OF DRAWINGS

FIG. 1 shows the structures of Compounds (1-1) to (1-12) as examples of the compound of the present invention.

FIG. 2 shows the structures of Compounds (1-13) to (1-24) as examples of the compound of the present invention.

FIG. 3 shows the structures of Compounds (1-25) to (1-36) as examples of the compound of the present invention.

FIG. 4 shows the structures of Compounds (1-37) to (1-45) as examples of the compound of the present invention.

FIG. 5 shows the structures of Compounds (1-46) to (1-50) as examples of the compound of the present invention.

FIG. 6 show an example of the structure of the organic EL element of the present invention.

### DESCRIPTION OF EMBODIMENTS

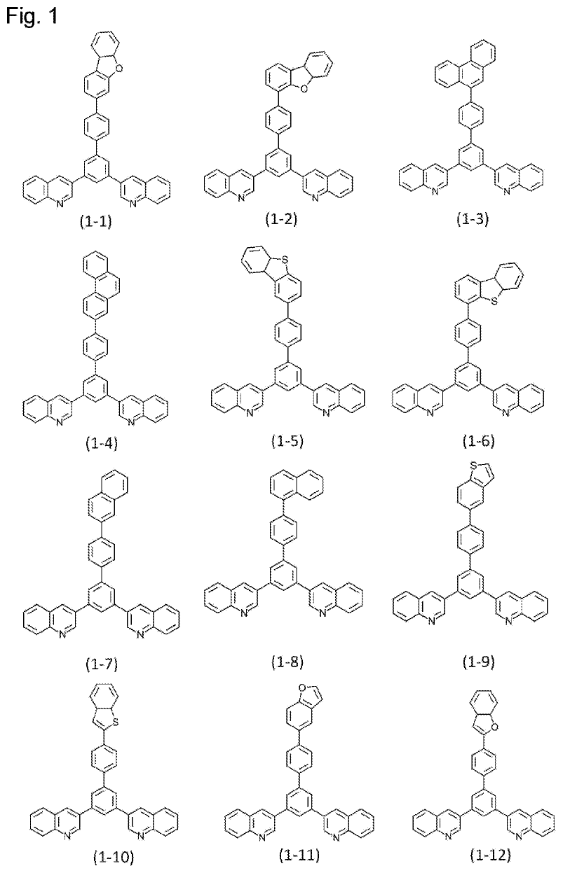

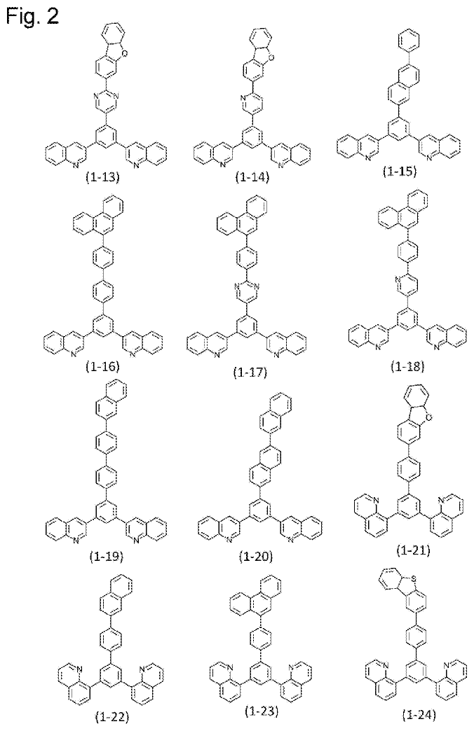

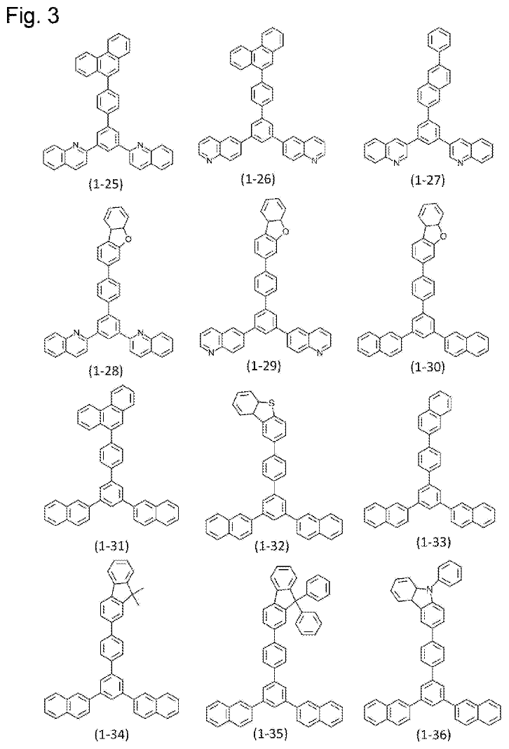

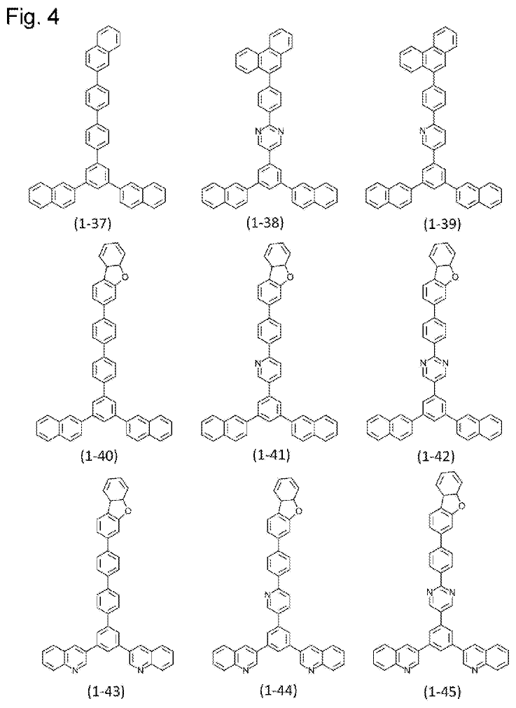

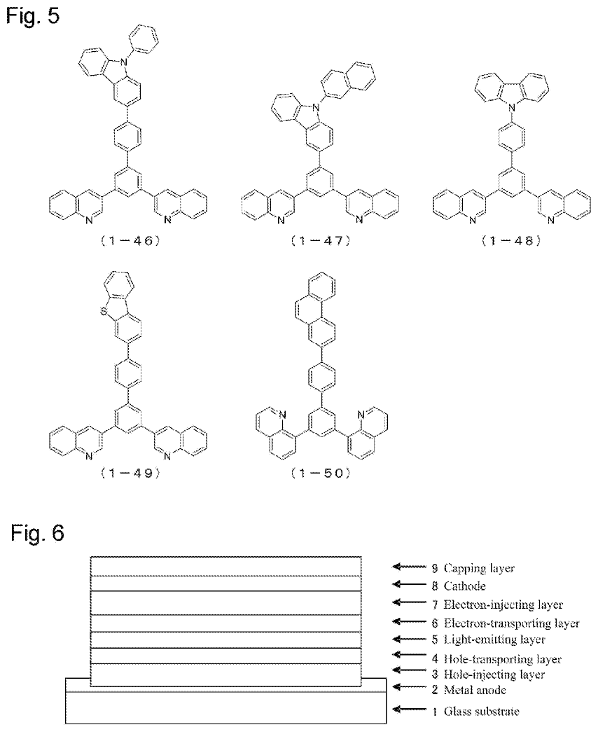

FIGS. 1 to 5 show specific examples of preferred compounds among those represented by the general formula (1) above of the present invention, but the invention is not limited to these compounds.

The compound represented by the general formula (1) above of the present invention is a novel compound, but can be synthesized according to known methods involving a cross-coupling reaction or the like.

There is no particular limitation on the method for purifying the compound represented by the general formula (1) above of the present invention, and any known methods for purification of organic compounds can be used, including column chromatography, adsorption using silica gel, activated carbon, activated clay, or the like, recrystallization and crystallization from a solvent, and sublimation. The compound can be identified by NMR analysis. The melting point, the glass transition point (Tg), and the refractive index are preferably measured as physical properties thereof.

The melting point and the glass transition point (Tg) can be measured on the compound in the form of a power using a high-sensitive differential scanning calorimeter (DSC3100SA manufactured by Bruker AXS K.K.).

The refractive index can be measured on an 80-nm thin film formed on a silicon substrate, using a spectroscopic analyzer (F10-RT-UV manufactured by Filmetrics Inc.).

The organic EL element of the present invention for use as a light-emitting element with a top emission structure may have a structure in which an anode, a hole-transporting layer, a light-emitting layer, an electron-transporting layer, a cathode, and a capping layer are sequentially provided on a glass substrate. The structure may further include any of a hole-injecting layer between the anode and the hole-transporting layer; an electron-blocking layer between the hole-transporting layer and the light-emitting layer; a hole-blocking layer between the light-emitting layer and the electron-transporting layer; and an electron-injecting layer between the electron-transporting layer and the cathode. In these multilayer structures, a single organic layer may perform the functions of some layers. For example, a single organic layer may serve as both the hole-injecting layer and the hole-transporting layer; both the hole-transporting layer and the electron-blocking layer; both the hole-blocking layer and the electron-transporting layer; or both the electron-transporting layer and the electron-injecting layer. It is also possible to stack two or more organic layers having the same function. Specifically, two hole-transporting layers may be stacked; two light-emitting layers may be stacked; two electron-transporting layers may be stacked; and two capping layers may be stacked, for example.

The total film thickness of the layers of the organic EL element is preferably approximately from 200 to 750 nm, and more preferably approximately from 350 to 600 nm. For example, the film thickness of the capping layer is preferably from 30 to 120 nm, and more preferably from 40 to 80 nm. This results in good light extraction efficiency. The film thickness of the capping layer may be tailored to the type of light-emitting material used for the light-emitting element, the thickness of the organic EL element excluding the capping layer, and others.

An electrode material having a high work function, such as ITO or gold, is used for the anode of the organic EL element of the present invention.

Examples of a material used for the hole-injecting layer of the organic EL element of the present invention include arylamine compounds having a molecular structure in which three or more triphenylamine structures are bonded to each other via a single bond or a hetero atom-free divalent group, such as starburst triphenylamine derivatives, and various triphenylamine tetramers; porphyrin compounds typified by copper phthalocyanine; heterocyclic compounds of acceptor type, such as hexacyanoazatriphenylene; and polymer materials of coating type.

Examples of a material used for the hole-transporting layer of the organic EL element of the present invention include benzidine derivatives such as N,N′-diphenyl-N,N′-di(m-tolyl)benzidine (hereinafter abbreviated as “TPD”), N,N′-diphenyl-N,N′-di(α-naphthyl)benzidine (hereinafter abbreviated as “NPD”), and N,N,N′,N′-tetrabiphenylyl benzidine; 1,1-bis[4-(di-4-tolylamino)phenyl]cyclohexane (hereinafter abbreviated as “TAPC”); and arylamine compounds having a molecular structure in which two triphenylamine structures are bonded to each other via a single bond or a hetero atom-free divalent in the molecule, such as, N,N,N′,N′-tetrabiphenylyl benzidine. Other examples include arylamine compounds having a molecular structure in which three or more triphenylamine structures are bonded to each other via a single bond or a hetero atom-free divalent group, such as various triphenylamine trimers and tetramers. It is also possible to use, as a material for the hole injecting/transporting layer, polymer materials of coating type, such as poly(3,4-ethylenedioxythiophene) (hereinafter abbreviated as “PEDOT”)/poly(styrenesulfonate) (hereinafter abbreviated as “PSS”).

Furthermore, other examples of a material used for the hole-injecting layer or the hole-transporting layer include those obtained by p-doping a material normally used for these layers with trisbromophenylamine hexachloroantimony or a radialene derivative (see Patent Literature 3, for example), and a polymer compound having the structure of a benzidine derivative, such as TPD, as a partial structure thereof.

Examples of a material used for the electron-blocking layer of the organic EL element of the present invention include compounds having an electron blocking effect, such as carbazole derivatives such as 4,4′,4″-tri(N-carbazolyl)triphenylamine (hereinafter abbreviated as “TCTA”), 9,9-bis[4-(carbazole-9-yl)phenyl]fluorene, 1,3-bis(carbazole-9-yl)benzene (hereinafter abbreviated as “mCP”), and 2,2-bis(4-carbazole-9-ylphenyl)adamantane (hereinafter abbreviated as “Ad-Cz”); and compounds having a triphenylsilyl group and a triarylamine structure and typified by 9-[4-(carbazole-9-yl)phenyl]-9-[4-(triphenylsilyl)phenyl]-9H-fluorene.

Examples of a material used for the light-emitting layer of the organic EL element of the present invention include light emitting materials, such as metal complexes of quinolinol derivatives such as Alq₃, various types of metal complexes, an anthracene derivative, a bisstyrylbenzene derivative, a pyrene derivative, an oxazole derivative, and a poly(p-phenylene vinylene) derivative. The light-emitting layer may also be composed of a host material and a dopant material. As the host material, an anthracene derivative is preferably used. Other examples of the host material include the above-listed light-emitting materials, and also heterocyclic compounds having an indole ring as a partial structure of a fused ring; heterocyclic compounds having a carbazole ring as a partial structure of a fused ring; and a carbazole derivative, a thiazole derivative, a benzimidazole derivative, and a polydialkylfluorene derivative. Examples of the dopant material include quinacridone, coumarin, rubrene, perylene, and derivatives thereof; a benzopyran derivative; a rhodamine derivative; and an aminostyryl derivative; and a green light-emitting material is preferably used.

A phosphorescent emitter can also be used as the light-emitting material. The phosphorescent emitter may be a complex of metal such as iridium or platinum, and examples thereof include a green phosphorescent emitter such as Ir(ppy)₃, a blue phosphorescent emitter such as FIrpic or FIr6, and a red phosphorescent emitter such as Btp₂Ir (acac). A green phosphorescent emitter is preferably used. As a host material in this case, a host material having hole-injecting/transporting capability may be used, including carbazole derivatives such as 4,4′-di(N-carbazolyl)biphenyl (hereinafter abbreviated as “CBP”), TCTA, and mCP, and a host material having electron-transporting capability may also be used, including p-bis(triphenylsilyl)benzene (hereinafter abbreviated as “UGH2”), and 2,2′,2″-(1,3,5-phenylene)-tris(1-phenyl-1H-benzimidazole) (hereinafter abbreviated as “TPBI”).

In order to avoid concentration quenching, doping of the host material with a phosphorescent material is preferably performed by co-deposition in an amount within a range of 1 to 30 wt % based on the entire light-emitting layer.

As the light-emitting material, materials that emit delayed fluorescence can also be used, including CDCB derivatives such as PIC-TRZ, CC2TA, PXZ-TRZ, and 4CzIPN (see Non-Patent Literature 4, for example).

Examples of a material used for the hole-blocking layer of the organic EL element of the present invention include compounds exhibiting a hole blocking effect, such as a phenanthroline derivative such as bathocuproine (hereinafter abbreviated as “BCP”), a metal complex of a quinolinol derivative, such as aluminum(III)bis(2-methyl-8-quinolinato)-4-phenylphenolate (hereinafter abbreviated as BAlq), various types of rare-earth complexes, a triazole derivative, a triazine derivative, a pyrimidine derivative, an oxadiazole derivative, and a benzoazole derivative. These materials may also serve as the material for the electron-transporting layer.

Examples of a material used for the electron-transporting layer of the organic EL element of the present invention include metal complexes of quinolinol derivatives such as Alq₃ and BAlq, various types of metal complexes, a triazole derivative, a triazine derivative, a pyrimidine derivative, an oxadiazole derivative, a pyridine derivative, a benzimidazole derivative, a benzoazole derivative, a thiadiazole derivative, an anthracene derivative, a carbodiimide derivative, a quinoxaline derivative, a pyridoindole derivative, a phenanthroline derivative, and a silole derivative.

Examples of a material used for the electron-injecting layer of the organic EL element of the present invention include alkali metal salts such as lithium fluoride and cesium fluoride, alkaline earth metal salts such as magnesium fluoride, metal complexes of quinolinol derivatives such as lithium quinolinol, metal oxides such as aluminum oxide, and metals such as ytterbium (Yb), samarium (Sm), calcium (Ca), strontium (Sr), and cesium (Cs). However, when an electron-transporting layer and a cathode are suitably selected, the electron-injecting layer can be omitted.

Furthermore, a material obtained by n-doping a material normally used for the electron-injecting layer or the electron-transporting layer with a metal such as cesium can be used for the electron-injecting layer or the electron-transporting layer.

Examples of an electrode material used for the cathode of the organic EL element of the present invention include an electrode material having a low work function, such as aluminum; an alloy having an even lower work function, such as a magnesium-silver alloy, a magnesium-calcium alloy, a magnesium-indium alloy, and an aluminum-magnesium alloy; and ITO and IZO.

An organic thin film containing the compound represented by the general formula (1) above is used for the capping layer of the organic EL element of the present invention.

The organic thin film containing the compound represented by the general formula (1) above and used for the capping layer preferably has a refractive index of 1.70 or more, and more preferably 1.85 or more, in a wavelength range of 450 to 750 nm, in view of improving the light extraction efficiency.

The above-described materials used for each of the layers constituting the organic EL element may be used singly for film formation, or two or more of these materials may be mixed and used for formation of a single layer. Any layer may have a layered structure composed of different layers each formed of a single kind of material, a layered structure composed of different layers each formed of a mixture of materials, or a layered structure composed of a layer formed of a single kind of material and a layer formed of a mixture of materials. These materials can be formed into a thin film by vapor deposition, or a known method such as spin coating or inkjet printing.

The present invention has been described hereinabove on the basis of an organic EL element with a top emission structure. However, the present invention is not limited thereto, and can also be applied in a similar manner to an organic EL element with a bottom emission structure or an organic EL element with a dual emission structure, which emits light from both of the top side and the bottom side. In these cases, it is necessary that the electrode positioned on the side of the light-emitting element from which light is extracted to the outside should be transparent or translucent.

Hereinafter, embodiments of the present invention will be described in greater detail by way of examples. However, the present invention is not limited to the examples below as long as it does not depart from the spirit thereof.

### EXAMPLES

### Example 1

### Synthesis of Example Compound (1-1)

12.5 g of 1,3-dibromo-5-chlorobenzene, 24.8 g of 3-quinolylboronic acid pinacol ester, 19.2 g of potassium carbonate, 130 ml of toluene, 40 ml of ethanol, and 40 ml of water were placed in a reaction vessel and mixed. 1.6 μg of tetrakis(triphenylphosphine)palladium(0) was added thereto, and the mixture was stirred overnight while heating under reflux. After the mixture was allowed to cool, dispersing and washing were performed at 80° C., and insoluble matters were filtered off. The filtrate was concentrated to obtain a crude product. The crude product was purified by crystallization from toluene and acetone, and the precipitated solid was collected to obtain 12.1 g of white powder of 3,3′-(5-chloro-1,3-phenylene)bis-quinoline (yield 71.34%).

Then, 5.0 g of 3,3′-(5-chloro-1,3-phenylene)bis-quinoline, 5.6 g of 3-[4-(4,4,5,5-tetramethyl-1,3,2-dioxaborolane-2-yl)phenyl]dibenzofuran, 5.8 μg of tripotassium phosphate, 50 ml of 1,4-dioxane, and 15 ml of water were placed in a reaction vessel and mixed. 0.4 μg of tris(dibenzylideneacetone)dipalladium(0) and 0.4 μg of tricyclohexylphosphine were added thereto, and the mixture was stirred overnight while heating under reflux. After the mixture was allowed to cool, water and methanol were added thereto, and the resulting mixture was stirred, followed by collecting the precipitated solid. The obtained solid was dispersed in monochlorobenzene at 100° C. for washing, and insoluble matters were filtered off. The filtrate was concentrated to obtain a crude product. The crude product was purified by crystallization from monochlorobenzene, and the precipitated solid was collected to obtain 5.1 g of white powder (yield 65.1%).

The structure of the obtained white powder was identified using NMR.

In ¹H-NMR (CDCl₃), the following signals of 26 hydrogens were detected, and thus it was seen that the obtained material was Example Compound (1-1).

δ (ppm)=9.34-9.34 (2H), 8.48-8.47 (2H), 8.21-8.19 (2H), 8.05-8.03 (4H), 8.00-7.94 (3H), 8.90-7.85 (5H), 7.79-7.78 (2H), 7.69-7.60 (4H), 7.51-7.46 (1H), 7.39-7.35 (1H).

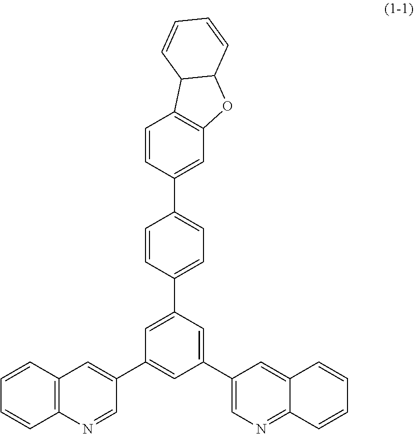 [SMILES: C1=CC2Oc3cc(-c4ccc(-c5cc(-c6cnc7ccccc7c6)cc(-c6cnc7ccccc7c6)c5)cc4)ccc3C2C=C1]

### Example 2

### Synthesis of Example Compound (1-3)

Synthesis was performed in the same manner as in Example 1 to thereby obtain 4.2 g of white powder (yield 57.4%), except that 4,4,5,5-tetramethyl-2-[4-(9-phenanthrenyl)phenyl]-1,3,2-dioxaborolane was used instead of 3-[4-(4,4,5,5-tetramethyl-1,3,2-dioxaborolane-2-yl)phenyl]dibenzofuran in Example 1.

The structure of the obtained white powder was identified using NMR.

In ¹H-NMR (CDCl₃), the following signals of 28 hydrogens were detected, and thus it was seen that the obtained powder was Example Compound (1-3).

δ (ppm)=9.37-9.36 (2H), 8.80 (2H), 8.51-8.50 (2H), 8.21-8.20 (2H), 8.10 (2H), 8.06-7.91 (7H), 7.80-7.26 (11H).

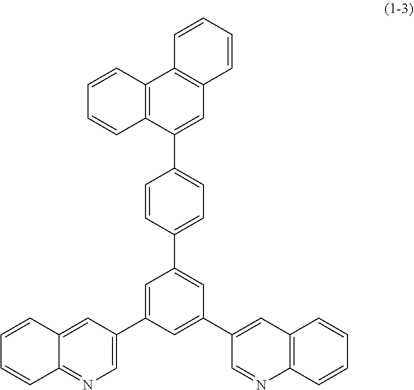 [SMILES: c1ccc2ncc(-c3cc(-c4ccc(-c5cc6ccccc6c6ccccc56)cc4)cc(-c4cnc5ccccc5c4)c3)cc2c1]

### Example 3

### Synthesis of Example Compound (1-49)

5.0 g of 3-(4-chlorophenyl)dibenzothiophene, 7.0 g of 3,3′-[5-(4,4,5,5-tetramethyl-1,3,2-dioxaborolane-2-yl)-1,3-phenylene]diquinoline, 9.7 g of potassium carbonate, 0.7 g of tris(dibenzylideneacetone)dipalladium(0), and 0.9 g of tricyclohexylphosphine were placed in a reaction vessel, and the mixture was stirred in 1,4-dioxane/H₂O mixed solvent overnight under reflux. After the mixture was allowed to cool, methanol was added thereto, and the resulting mixture was stirred, followed by collecting the precipitated solid. The obtained solid was dispersed in monochlorobenzene at 100° C. for washing, and insoluble matters were filtered off. The filtrate was concentrated to obtain a crude product. The crude product was purified by crystallization from monochlorobenzene, and the precipitated solid was collected to obtain 5.8 g of white powder (yield 64.3%).

The structure of the obtained white powder was identified using NMR.

In ¹H-NMR (CDCl₃), the following signals of 26 hydrogens were detected, and thus it was seen that the obtained powder was Example Compound (1-49).

δ (ppm)=9.36-9.35 (2H), 8.50 (2H), 8.23 (1H), 8.22-8.16 (4H), 8.06-8.04 (3H), 7.97-7.96 (2H), 7.90-7.88 (5H), 7.81-7.76 (3H), 7.66-7.64 (2H), 7.50-7.48 (2H).

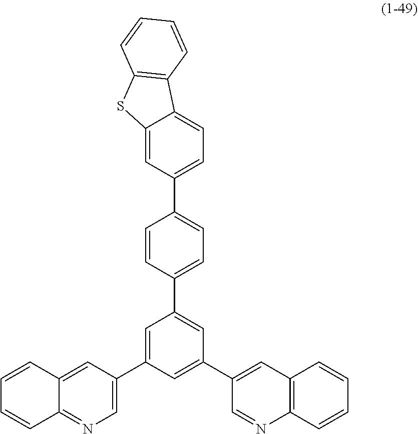 [SMILES: c1ccc2ncc(-c3cc(-c4ccc(-c5ccc6c(c5)sc5ccccc56)cc4)cc(-c4cnc5ccccc5c4)c3)cc2c1]

### Example 4

### Synthesis of Example Compound (1-46)

5.0 g of 3-(4-bromophenyl)-9-phenyl-9H-carbazole, 6.4 g of 3,3′-[5-(4,4,5,5-tetramethyl-1,3,2-dioxaborolane-2-yl)-1,3-phenylene]diquinoline, 3.5 g of potassium carbonate, and 0.3 g of tetrakis(triphenylphosphine)palladium(0) were placed in a reaction vessel, and the mixture was stirred in toluene/EtOH/H₂O mixed solvent overnight under reflux. After the mixture was allowed to cool, methanol was added thereto, and the resulting mixture was stirred, followed by collecting the precipitated solid. The obtained solid was dispersed in toluene at 100° C. for washing, and insoluble matters were filtered off. The filtrate was concentrated to obtain a crude product. The crude product was purified by crystallization from toluene and acetone, and the precipitated solid was collected to obtain 3.9 g of white powder (yield 47.8%).

The structure of the obtained white powder was identified using NMR.

In ¹H-NMR (CDCl₃), the following signals of 31 hydrogens were detected, and thus it was seen that the obtained powder was Example Compound (1-46).

δ (ppm)=9.35 (2H), 8.48-8.44 (3H), 8.23-8.19 (3H), 8.06-8.02 (3H), 7.96 (2H), 7.92-7.87 (4H), 7.80-7.73 (3H), 7.66-7.60 (6H), 7.51-7.48 (2H), 7.44-7.43 (2H), 7.35-7.32 (1H).

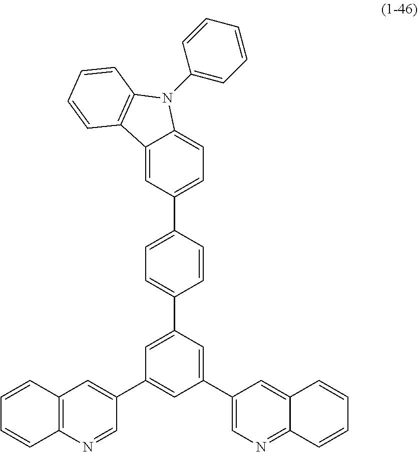 [SMILES: c1ccc(-n2c3ccccc3c3cc(-c4ccc(-c5cc(-c6cnc7ccccc7c6)cc(-c6cnc7ccccc7c6)c5)cc4)ccc32)cc1]

### Example 5

### Synthesis of Example Compound (1-47)

5.0 g of 3-bromo-9-(2-naphthalenyl)-9H-carbazole, 6.5 g of 3,3′-[5-(4,4,5,5-tetramethyl-1,3,2-dioxaborolane-2-yl)-1,3-phenylene]diquinoline, 3.7 g of potassium carbonate, and 0.3 g of tetrakis(triphenylphosphine)palladium(0) were placed in a reaction vessel, and the mixture was stirred in toluene/EtOH/H₂O mixed solvent overnight under reflux. After the mixture was allowed to cool, methanol was added thereto, and the resulting mixture was stirred, followed by collecting the precipitated solid. The obtained solid was dispersed in toluene at 100° C. for washing, and insoluble matters were filtered off. The filtrate was concentrated to obtain a crude product. The crude product was purified by crystallization from toluene and acetone, and the precipitated solid was collected to obtain 4.1 g of white powder (yield 48.9%).

The structure of the obtained white powder was identified using NMR.

In ¹H-NMR (CDCl₃), the following signals of 29 hydrogens were detected, and thus it was seen that the obtained powder was Example Compound (1-47).

δ (ppm)=9.37 (2H), 8.54-8.50 (3H), 8.28-8.26 (1H), 8.21-8.19 (2H), 8.12-8.10 (4H), 8.01-7.93 (5H), 7.83-7.70 (4H), 7.64-7.58 (5H), 7.51-7.46 (2H), 7.38-7.34 (1H).

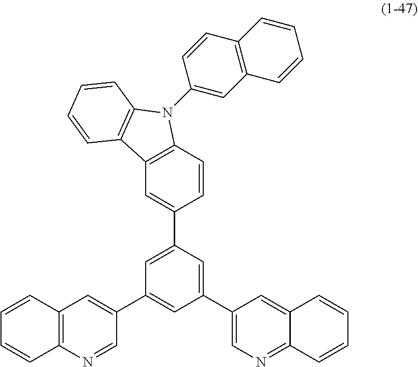 [SMILES: c1ccc2cc(-n3c4ccccc4c4cc(-c5cc(-c6cnc7ccccc7c6)cc(-c6cnc7ccccc7c6)c5)ccc43)ccc2c1]

### Example 6

### Synthesis of Example Compound (1-48)

6.0 g of 9-(4-bromophenyl)-9H-carbazole, 9.0 g of 3,3′-[5-(4,4,5,5-tetramethyl-1,3,2-dioxaborolane-2-yl)-1,3-phenylene]diquinoline, 5.2 g of potassium carbonate, and 0.4 g of tetrakis(triphenylphosphine)palladium(0) were placed in a reaction vessel, and the mixture was stirred in toluene/EtOH/H₂O mixed solvent overnight under reflux. After the mixture was allowed to cool, methanol was added thereto, and the resulting mixture was stirred, followed by collecting the precipitated solid. The obtained solid was dispersed in monochlorobenzene at 100° C. for washing, and insoluble matters were filtered off. The filtrate was concentrated to obtain a crude product. The crude product was purified by crystallization from monochlorobenzene and acetone, and the precipitated solid was collected to obtain 6.8 g of white powder (yield 63.7%).

The structure of the obtained white powder was identified using NMR.

In ¹H-NMR (CDCl₃), the following signals of 27 hydrogens were detected, and thus it was seen that the obtained powder was Example Compound (1-48).

δ (ppm)=9.37 (2H), 8.51-8.50 (2H), 8.22-8.16 (4H), 8.08-8.07 (3H), 8.01-7.95 (4H), 7.81-7.75 (4H), 7.66-7.62 (2H), 7.53-7.51 (2H), 7.47-7.43 (2H), 7.34-7.30 (2H).

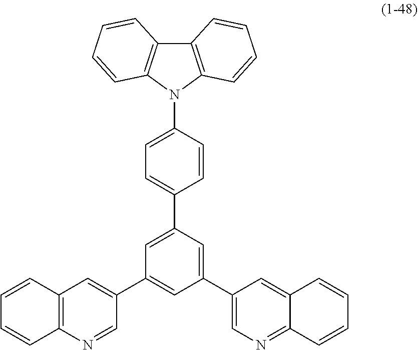 [SMILES: c1ccc2ncc(-c3cc(-c4ccc(-n5c6ccccc6c6ccccc65)cc4)cc(-c4cnc5ccccc5c4)c3)cc2c1]

### Example 7

### Synthesis of Example Compound (1-17)

5.0 g of 5-chloro-2-(4-phenanthrene-9-yl-phenyl)-pyrimidine, 6.9 g of 1,3-bis(quinoline-3-yl)-5-(4,4,5,5-tetramethyl-1,3,2-dioxaborolane-2-yl)-benzene, 2.8 g of potassium carbonate, and 0.4 g of tetrakis(triphenylphosphine)palladium(0) were placed in a reaction vessel, and the mixture was stirred in toluene/EtOH/H₂O mixed solvent overnight under reflux. After the mixture was allowed to cool, methanol was added thereto, and the resulting mixture was stirred, followed by collecting the precipitated solid. The obtained solid was dispersed in toluene at 100° C. for washing, and insoluble matters were filtered off. The filtrate was concentrated to obtain a crude product. The crude product was purified by crystallization from monochlorobenzene, and the precipitated solid was collected to obtain 4.3 g of white powder (yield 47.6%).

The structure of the obtained white powder was identified using NMR.

In ¹H-NMR (CDCl₃), the following signals of 30 hydrogens were detected, and thus it was seen that the obtained powder was Example Compound (1-17).

δ (ppm)=9.34 (2H), 9.24 (2H), 8.80 (1H), 8.74 (1H), 8.69 (2H), 8.49 (2H), 8.21 (2H), 8.12 (1H), 8.03 (2H), 8.00 (1H), 7.97 (2H), 7.93 (1H), 7.80 (2H), 7.77 (1H), 7.74 (2H), 7.72-7.60 (5H), 7.57 (1H).

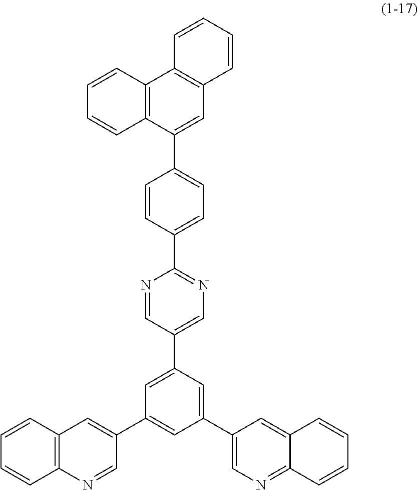 [SMILES: c1ccc2ncc(-c3cc(-c4cnc(-c5ccc(-c6cc7ccccc7c7ccccc67)cc5)nc4)cc(-c4cnc5ccccc5c4)c3)cc2c1]

### Example 8

### Synthesis of Example Compound (1-18)

4.5 g of 5-bromo-2-(4-phenanthrene-9-yl-phenyl)-pyridine, 5.5 g of 1,3-bis(quinoline-3-yl)-5-(4,4,5,5-tetramethyl-1,3,2-dioxaborolane-2-yl)-benzene, 2.3 g of potassium carbonate, and 0.3 g of tetrakis(triphenylphosphine)palladium(0) were placed in a reaction vessel, and the mixture was stirred in toluene/EtOH/H₂O mixed solvent overnight under reflux. After the mixture was allowed to cool, methanol was added thereto, and the resulting mixture was stirred, followed by collecting the precipitated solid. The obtained solid was dispersed in monochlorobenzene solvent at 100° C. for washing, and insoluble matters were filtered off. The filtrate was concentrated to obtain a crude product. The crude product was purified by column chromatography (stationary phase: silica gel, eluent: ethyl acetate/dichloroethane) to obtain 5.3 g of white powder (yield 73.0%).

The structure of the obtained white powder was identified using NMR.

In ¹H-NMR (CDCl₃), the following signals of 31 hydrogens were detected, and thus it was seen that the obtained powder was Example Compound (1-18).

δ (ppm)=9.35 (2H), 8.16 (1H), 8.80 (1H), 8.74 (1H), 8.48 (2H), 8.25 (2H), 8.21 (2H), 8.17 (1H), 8.08 (1H), 8.05 (2H), 8.01 (2H), 7.96 (2H), 7.93 (1H), 7.79 (2H), 7.76 (1H), 7.74-7.60 (7H), 7.57 (1H).

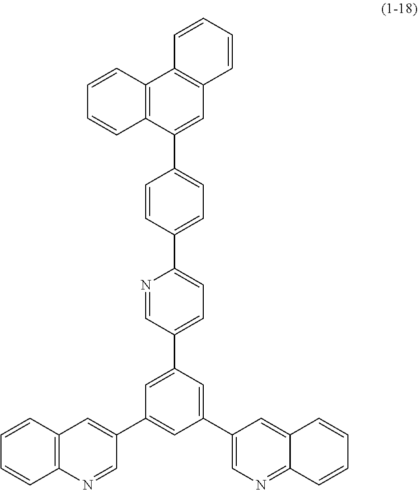 [SMILES: c1ccc2ncc(-c3cc(-c4ccc(-c5ccc(-c6cc7ccccc7c7ccccc67)cc5)nc4)cc(-c4cnc5ccccc5c4)c3)cc2c1]

### Example 9

### Synthesis of Example Compound (1-21)

12.5 g of 1,3-dibromo-5-chlorobenzene, 16.8 g of 8-quinolineboronic acid, 19.2 g of potassium carbonate, 130 ml of toluene, 40 ml of ethanol, and 40 ml of water were placed in a reaction vessel and mixed. 1.6 g of tetrakis(triphenylphosphine)palladium(0) was added thereto, and the mixture was stirred overnight while heating under reflux. The resulting mixture was allowed to cool, and then subjected to extraction with toluene. The organic layer was dispersed and washed at 80° C., and the filtrate was concentrated to obtain a crude product. The crude product was purified by crystallization from acetone, and the precipitated solid was collected to obtain 12.1 g of white powder of 8,8′-(5-chloro-1,3-phenylene)bis-quinoline (yield 71.34%).

Then, 5.7 g of 8,8′-(5-chloro-1,3-phenylene)bis-quinoline, 6.3 g of 3-[4-(4,4,5,5-tetramethyl-1,3,2-dioxaborolane-2-yl)phenyl]dibenzofuran, 6.6 g of tripotassium phosphate, 60 ml of 1,4-dioxane, and 18 ml of water were placed in a reaction vessel and mixed. 0.4 g of tris(dibenzylideneacetone)dipalladium(0) and 0.4 g of tricyclohexylphosphine were added thereto, and the mixture was stirred overnight while heating under reflux. After the mixture was allowed to cool, water and methanol were added thereto, and the resulting mixture was stirred, followed by collecting the precipitated solid. The obtained solid was dispersed in monochlorobenzene at 100° C. for washing, and insoluble matters were filtered off. The filtrate was concentrated to obtain a crude product. The crude product was purified by crystallization from monochlorobenzene, and the precipitated solid was collected to obtain 6.7 g of white powder (yield 75.0%).

The structure of the obtained white powder was identified using NMR.

In ¹H-NMR (CDCl₃), the following signals of 26 hydrogens were detected, and thus it was seen that the obtained powder was Example Compound (1-21).

δ (ppm)=9.00-8.99 (2H), 8.24-8.21 (2H), 8.08-7.94 (7H), 7.89-7.85 (5H), 7.79-7.77 (2H), 7.70-7.63 (3H), 7.60-7.58 (1H), 7.48-7.42 (3H), 7.37-7.34 (1H).

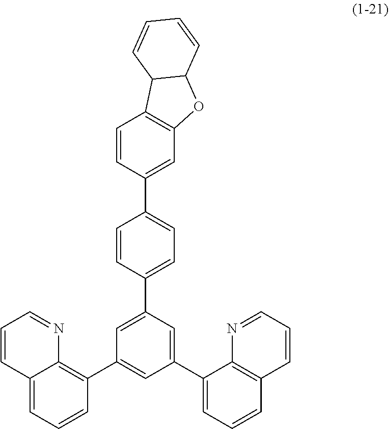 [SMILES: C1=CC2Oc3cc(-c4ccc(-c5cc(-c6cccc7cccnc67)cc(-c6cccc7cccnc67)c5)cc4)ccc3C2C=C1]

### Example 10

### Synthesis of Example Compound (1-22)

Synthesis was performed in the same manner as in Example 9 to obtain 3.2 g of white powder (yield 54.9%), except that 4-(2-naphthyl)phenylboronic acid was used instead of 3-[4-(4,4,5,5-tetramethyl-1,3,2-dioxaborolane-2-yl)phenyl]dibenzofuran in Example 9.

The structure of the obtained white powder was identified using NMR.

In ¹H-NMR (CDCl₃), the following signals of 26 hydrogens were detected, and thus it was seen that the obtained powder was Example Compound (1-22).

δ (ppm)=9.00-8.98 (2H), 8.24-8.21 (2H), 8.10-8.08 (3H), 8.04 (1H), 7.96-7.80 (12H), 7.67-7.63 (2H), 7.52-7.47 (2H), 7.46-7.42 (2H).

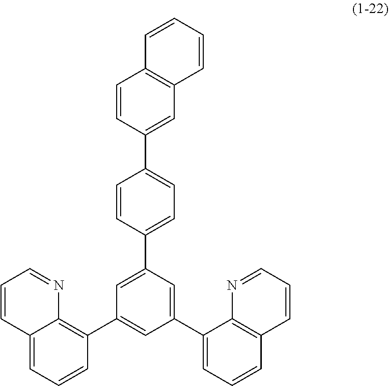 [SMILES: c1ccc2cc(-c3ccc(-c4cc(-c5cccc6cccnc56)cc(-c5cccc6cccnc56)c4)cc3)ccc2c1]

### Example 11

### Synthesis of Example Compound (1-23)

Synthesis was performed in the same manner as in Example 9 to obtain 8.8 g of white powder (yield 55.0%), except that 4,4,5,5-tetramethyl-2-[4-(9-phenanthrenyl)phenyl]-1,3,2-dioxaborolane was used instead of 3-[4-(4,4,5,5-tetramethyl-1,3,2-dioxaborolane-2-yl)phenyl]dibenzofuran in Example 9.

The structure of the obtained white powder was identified using NMR.

In ¹H-NMR (CDCl₃), the following signals of 28 hydrogens were detected, and thus it was seen that the obtained powder was Example Compound (1-23). δ (ppm)=9.01 (2H), 8.78-8.70 (2H), 8.22-8.19 (2H), 8.13-8.02 (4H), 7.97-7.83 (7H), 7.74 (1H1), 7.68-7.53 (8H1), 7.40 (2H1).

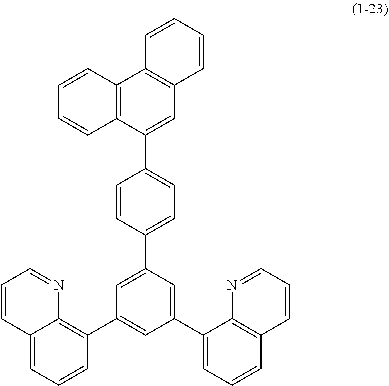 [SMILES: c1cnc2c(-c3cc(-c4ccc(-c5cc6ccccc6c6ccccc56)cc4)cc(-c4cccc5cccnc45)c3)cccc2c1]

### Example 12

### Synthesis of Example Compound (1-24)

Synthesis was performed in the same manner as in Example 9 to obtain 6.7 g of white powder (yield 46.0%), except that 2-[4-(4,4,5,5-tetramethyl-1,3,2-dioxaborolane-2-yl)phenyl]dibenzo thiophene was used instead of 3-[4-(4,4,5,5-tetramethyl-1,3,2-dioxaborolane-2-yl)phenyl]dibenzofuran in Example 9.

The structure of the obtained white powder was identified using NMR.

In ¹H-NMR (CDCl₃), the following signals of 26 hydrogens were detected, and thus it was seen that the obtained powder was Example Compound (1-24).

δ (ppm)=9.00 (2H), 8.40 (1H), 8.23-8.20 (3H), 8.09-8.05 (3H), 7.96-7.74 (11H), 7.66-7.62 (2H), 7.47-7.40 (4H).

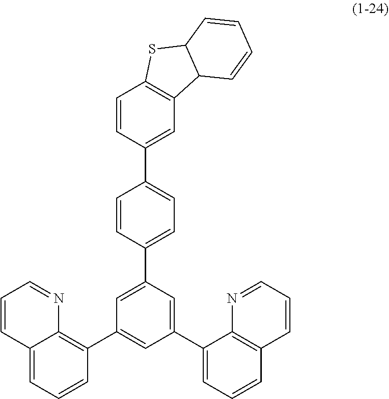 [SMILES: C1=CC2Sc3ccc(-c4ccc(-c5cc(-c6cccc7cccnc67)cc(-c6cccc7cccnc67)c5)cc4)cc3C2C=C1]

### Example 13

### Synthesis of Example Compound (1-50)

Synthesis was performed in the same manner as in Example 9 to obtain 11.0 g of white powder (yield 72.8%), except that 4,4,5,5-tetramethyl-2-[4-(phenanthrene-2-yl)phenyl]-1,3,2-dioxaborolane was used instead of 3-[4-(4,4,5,5-tetramethyl-1,3,2-dioxaborolane-2-yl)phenyl]dibenzofuran in Example 9.

The structure of the obtained white powder was identified using NMR.

In ¹H-NMR (CDCl₃), the following signals of 28 hydrogens were detected, and thus it was seen that the obtained powder was Example Compound (1-50).

δ (Ppm)=9.00-8.99 (2H), 8.77-8.75 (1H), 8.72-8.70 (1H), 8.24-8.22 (2H), 8.16 (1H), 8.09-8.08 (2H), 8.04-8.03 (1H), 7.99-7.94 (3H), 7.91-7.85 (7H), 7.83-7.76 (2H), 7.69-7.62 (3H), 7.62-7.58 (1H), 7.45-7.42 (2H).

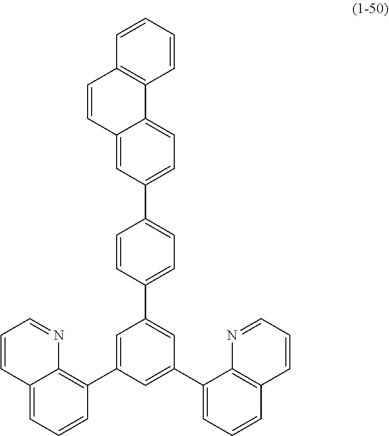 [SMILES: c1cnc2c(-c3cc(-c4ccc(-c5ccc6c(ccc7ccccc76)c5)cc4)cc(-c4cccc5cccnc45)c3)cccc2c1]

### Example 14

### Synthesis of Example Compound (1-33)

20.8 g of 1,3-dibromo-5-chlorobenzene, 29.1 g of 2-naphthaleneboronic acid, 31.9 g of potassium carbonate, 200 ml of toluene, 60 ml of ethanol, and 60 ml of water were placed in a reaction vessel and mixed. 2.6 g of tetrakis(triphenylphosphine)palladium(0) was added thereto, and the mixture was stirred overnight while heating under reflux. The mixture was allowed to cool, and then subjected to extraction with toluene. The organic layer was dispersed and washed at 80° C., and the filtrate was concentrated to obtain a crude product. The crude product was purified by crystallization from toluene and acetone, and the precipitated solid was collected to obtain 24.0 g (yield 85.5%) of white powder of 2,2′-(5-chloro-1,3-phenylene)bis-naphthalene.

Then, 10.0 g of 2,2′-(5-chloro-1,3-phenylene)bis-naphthalene, 7.5 g of 4-(2-naphthyl)phenylboronic acid, 11.6 g of tripotassium phosphate, 100 ml of 1,4-dioxane, and 30 ml of water were placed in a reaction vessel and mixed. 0.8 g of tris(dibenzylideneacetone)dipalladium(0) and 0.8 g of tricyclohexylphosphine were added thereto, and the mixture was stirred overnight while heating under reflux. After the mixture was allowed to cool, water and methanol were added thereto, and the resulting mixture was stirred, followed by collecting the precipitated solid. The obtained solid was dispersed in toluene at 80° C. for washing, and insoluble matters were filtered off. The filtrate was concentrated to obtain a crude product. The crude product was purified by crystallization from toluene, and the precipitated solid was collected to obtain 10.5 g of white powder (yield 71.9%).

The structure of the obtained white powder was identified using NMR.

In ¹H-NMR (CDCl₃), the following signals of 28 hydrogens were detected, and thus it was seen that the obtained powder was Example Compound (1-33).

δ (ppm)=8.20 (2H), 8.12 (1H), 8.06 (1H), 8.01-7.99 (3H), 7.97-7.94 (5H), 7.91-7.88 (9H), 7.83-7.81 (1H), 7.56-7.47 (6H).

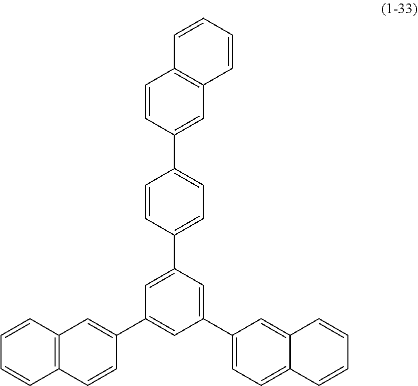 [SMILES: c1ccc2cc(-c3ccc(-c4cc(-c5ccc6ccccc6c5)cc(-c5ccc6ccccc6c5)c4)cc3)ccc2c1]

### Example 15

### Synthesis of Example Compound (1-31)

Synthesis was performed in the same manner as in Example 14 to obtain 11.4 g of white powder (yield 79.3%), except that 4,4,5,5-tetramethyl-2-[4-(9-phenanthrenyl)phenyl]-1,3,2-dioxaborolane was used instead of 4-(2-naphthyl)phenylboronic acid in Example 14.

The structure of the obtained white powder was identified using NMR.

In ¹H-NMR (CDCl₃), the following signals of 30 hydrogens were detected, and thus it was seen that the obtained powder was Example Compound (1-31).

δ (ppm)=8.82-8.80 (1H), 8.76-8.74 (1H), 8.23 (2H), 8.09-7.95 (7H), 7.94-7.90 (8H), 7.77 (1H), 7.72-7.68 (3H), 7.67-7.50 (7H).

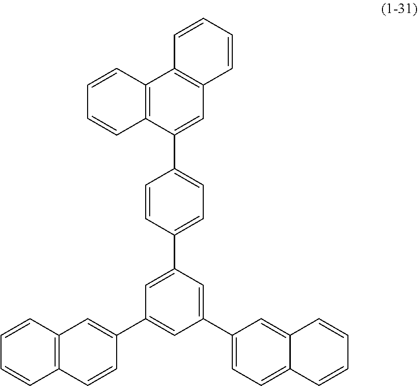 [SMILES: c1ccc2cc(-c3cc(-c4ccc(-c5cc6ccccc6c6ccccc56)cc4)cc(-c4ccc5ccccc5c4)c3)ccc2c1]

### Example 16

### Synthesis of Example Compound (1-34)

Synthesis was performed in the same manner as in Example 14 to obtain 3.3 g of white powder (yield 42.0%), except that 2-[4-(9,9-dimethyl-9H-fluorene-2-yl)phenyl]-4,4,5,5-tetramethyl-1,3,2-dioxaborolane was used instead of 4-(2-naphthyl)phenylboronic acid in Example 14.

The structure of the obtained white powder was identified using NMR.

In ¹H-NMR (CDCl₃), the following signals of 34 hydrogens were detected, and thus it was seen that the obtained powder was Example Compound (1-34).

δ (ppm)=8.20 (2H), 8.06 (1H), 8.01-7.95 (6H), 7.92-7.87 (6H), 7.83-7.81 (3H), 7.75 (1H), 7.73 (1H), 7.67 (1H), 7.55-7.52 (4H), 7.46 (1H), 7.36-7.35 (2H), 1.57 (6H).

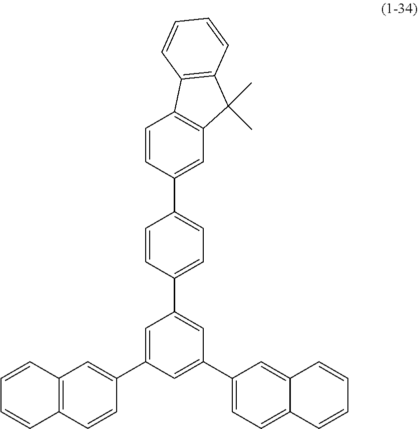 [SMILES: CC1(C)c2ccccc2-c2ccc(-c3ccc(-c4cc(-c5ccc6ccccc6c5)cc(-c5ccc6ccccc6c5)c4)cc3)cc21]

### Example 17

### Synthesis of Example Compound (1-35)

Synthesis was performed in the same manner as in Example 14 to obtain 5.9 g of white powder (yield 79.0%), except that 2-[4-(9,9-diphenyl-9H-fluorene-2-yl)phenyl]-4,4,5,5-tetramethyl-1,3,2-dioxaborolane was used instead of 4-(2-naphthyl)phenylboronic acid in Example 14.

The structure of the obtained white powder was identified using NMR.

In ¹H-NMR (CDCl₃), the following signals of 38 hydrogens were detected, and thus it was seen that the obtained powder was Example Compound (1-35).

δ (ppm)=8.18 (2H), 8.04 (1H), 7.99-7.84 (11H), 7.82-7.79 (3H), 7.71-7.67 (4H), 7.54-7.51 (4H), 7.44-7.42 (1H), 7.40-7.36 (1H), 7.29-7.22 (11H).

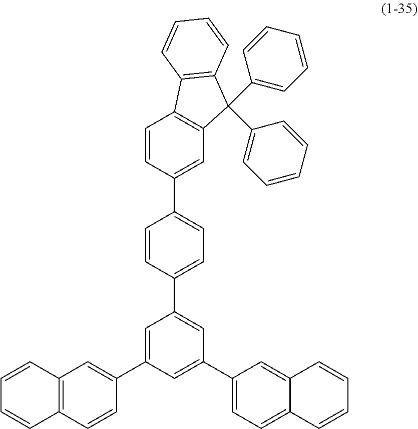 [SMILES: c1ccc(C2(c3ccccc3)c3ccccc3-c3ccc(-c4ccc(-c5cc(-c6ccc7ccccc7c6)cc(-c6ccc7ccccc7c6)c5)cc4)cc32)cc1]

### Example 18

### Synthesis of Example Compound (1-30)

Synthesis was performed in the same manner as in Example 14 to obtain 9.5 g of white powder (yield 77.0%), except that 2-[4-(dibenzo furan-3-yl)phenyl]-4,4,5,5-tetramethyl-1,3,2-dioxaborolane was used instead of 4-(2-naphthyl)phenylboronic acid in Example 14.

The structure of the obtained white powder was identified using NMR.

In ¹H-NMR (CDCl₃), the following signals of 28 hydrogens were detected, and thus it was seen that the obtained powder was Example Compound (1-30).

δ (ppm)=8.21 (2H), 8.07-8.06 (1H), 8.05-7.97 (7H), 7.95 (1H), 7.92-7.83 (9H), 7.70-7.67 (1H1), 7.62-7.60 (1H1), 7.56-7.48 (5H1), 7.39-7.37 (1H1).

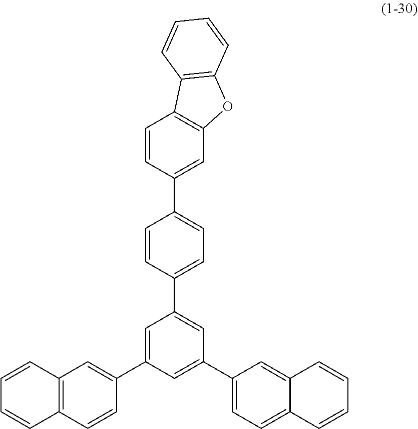 [SMILES: c1ccc2cc(-c3cc(-c4ccc(-c5ccc6c(c5)oc5ccccc56)cc4)cc(-c4ccc5ccccc5c4)c3)ccc2c1]

### Example 19

### Synthesis of Example Compound (1-36)

Synthesis was performed in the same manner as in Example 14 to obtain 11.1 g of white powder (yield 73.5%), except that 9-phenyl-3-[4-(4,4,5,5-tetramethyl-1,3,2-dioxaborolane-2-yl)phenyl]-9H-carbazole was used instead of 4-(2-naphthyl)phenylboronic acid in Example 14.

The structure of the obtained white powder was identified using NMR.

In ¹H-NMR (CDCl₃), the following signals of 33 hydrogens were detected, and thus it was seen that the obtained powder was Example Compound (1-36).

δ (ppm)=8.44 (1H), 8.24-8.22 (3H), 8.06-8.05 (1H), 8.03-7.95 (6H), 7.93-7.88 (8H), 7.75-7.72 (1H), 7.65-7.60 (4H), 7.56-7.49 (6H), 7.45-7.44 (2H), 7.34-7.33 (1H).

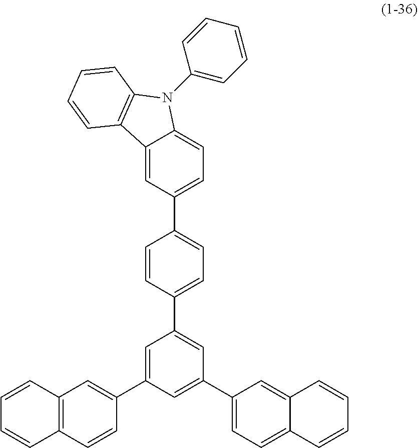 [SMILES: c1ccc(-n2c3ccccc3c3cc(-c4ccc(-c5cc(-c6ccc7ccccc7c6)cc(-c6ccc7ccccc7c6)c5)cc4)ccc32)cc1]

### Example 20

### Synthesis of Example Compound (1-38)

4.5 g of 5-chloro-2-(4-phenanthrene-9-yl-phenyl)-pyrimidine, 6.7 g of 2-[3,5-di(naphthalene-2-yl)phenyl]-4,4,5,5-tetramethyl-1,3,2-dioxaborolane-, 2.5 g of potassium carbonate, and 0.4 g of tetrakis(triphenylphosphine)palladium(0) were placed in a reaction vessel, and the mixture was stirred in toluene/EtOH/H₂O mixed solvent overnight under refluxed. After the mixture was allowed to cool, methanol was added thereto, and the resulting mixture was stirred, followed by collecting the precipitated solid. The obtained solid was dispersed in monochlorobenzene at 100° C. for washing, and insoluble matters were filtered off. The filtrate was concentrated to obtain a crude product. The crude product was purified by crystallization from monochlorobenzene, and the precipitated solid was collected to obtain 1.8 g of white powder (yield 22.2%).

The structure of the obtained white powder was identified using NMR.

In ¹H-NMR (CDCl₃), the following signals of 32 hydrogens were detected, and thus it was seen that the obtained powder was Example Compound (1-38).

δ (ppm)=9.24 (2H), 8.81-8.79 (1H), 8.76-8.74 (1H), 8.70-8.67 (2H), 8.22 (2H), 8.16-8.15 (1H), 8.03-7.97 (7H), 7.94-7.89 (5H), 7.77-7.70 (5H), 7.68-7.64 (1H), 7.59-7.54 (5H).

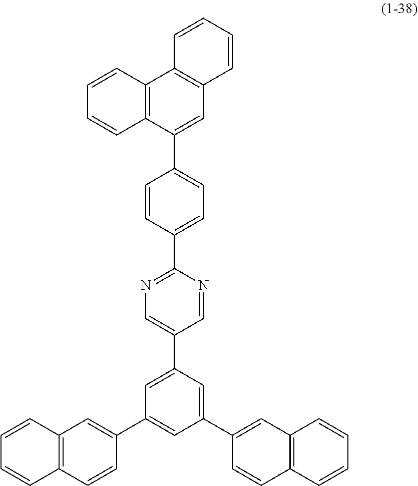 [SMILES: c1ccc2cc(-c3cc(-c4cnc(-c5ccc(-c6cc7ccccc7c7ccccc67)cc5)nc4)cc(-c4ccc5ccccc5c4)c3)ccc2c1]

### Example 21

### Synthesis of Example Compound (1-39)

5.0 g of 5-bromo-2-(4-phenanthrene-9-yl-phenyl)-pyridine, 6.7 g of 2-[3,5-di(naphthalene-2-yl)phenyl]-4,4,5,5-tetramethyl-1,3,2-dioxaborolane-, 2.5 g of potassium carbonate, and 0.4 g of tetrakis(triphenylphosphine)palladium(0) were placed in a reaction vessel, and the mixture was stirred in toluene/EtOH/H₂O mixed solvent overnight under reflux. After the mixture was allowed to cool, methanol was added thereto, and the resulting mixture was stirred, followed by collecting the precipitated solid. The obtained solid was dispersed in monochlorobenzene at 100° C. for washing, and insoluble matters were filtered off. The filtrate was concentrated to obtain a crude product. The crude product was purified by crystallization from toluene, and the precipitated solid was collected to obtain 6.7 g of white powder (yield 83.3%).

The structure of the obtained white powder was identified using NMR.

In ¹H-NMR (CDCl₃), the following signals of 33 hydrogens were detected, and thus it was seen that the obtained powder was Example Compound (1-39).

δ (ppm)=9.16 (1H), 8.81-8.79 (1H), 8.75-8.73 (1H), 8.25-8.21 (4H), 8.18-8.15 (1H), 8.12-8.11 (1H), 8.02-7.96 (8H), 7.93-7.89 (5H), 7.76 (1H), 7.72-7.67 (4H), 7.63-7.61 (1H), 7.58-7.51 (5H).

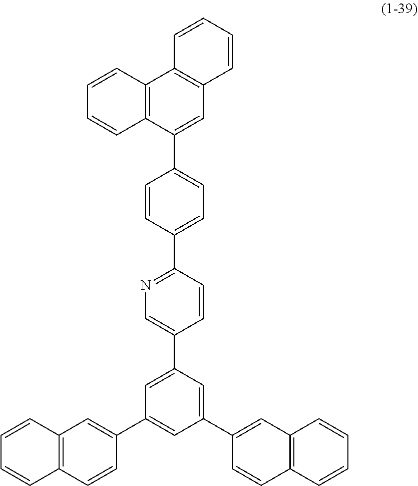 [SMILES: c1ccc2cc(-c3cc(-c4ccc(-c5ccc(-c6cc7ccccc7c7ccccc67)cc5)nc4)cc(-c4ccc5ccccc5c4)c3)ccc2c1]

### Example 22

### Synthesis of Example Compound (1-25)

16.0 g of 1-bromo-3,5-dichlorobenzene, 28.3 g of 4,4,5,5-tetramethyl-2-[4-(9-phenanthrenyl)phenyl]-1,3,2-dioxaborolane, 19.6 g of potassium carbonate, 200 ml of toluene, 60 ml of ethanol, and 60 ml of water were placed in a reaction vessel and mixed. 1.6 g of tetrakis(triphenylphosphine)palladium(0) was added thereto, and the mixture was stirred overnight while heating under reflux. The mixture was allowed to cool, and then subjected to extraction with toluene. The organic layer was dispersed and washed at 80° C., and the filtrate was concentrated to obtain a crude product. The crude product was purified by crystallization from toluene and acetone, and the precipitated solid was collected to obtain 21.2 g of white solid of phenanthrene derivative represented by the formula (I-1) below (yield 75.0%).

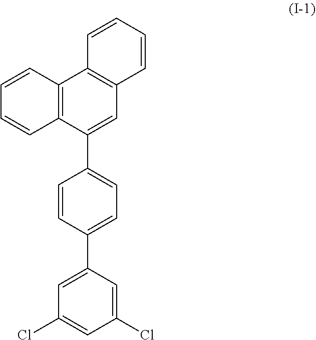 [SMILES: Clc1cc(Cl)cc(-c2ccc(-c3cc4ccccc4c4ccccc34)cc2)c1]

Then, 20.0 g of the phenanthrene derivative represented by the formula (I-1) above, 28.0 g of bis(pinacolato)diboron, 14.8 g of potassium acetate, and 200 ml of N—N dimethylformamide were placed in a reaction vessel and mixed. 1.7 g of dichloro[1,1′-bis(diphenylphosphino)ferrocene]palladium and 5.6 g of tricyclohexylphosphine were added thereto, and the mixture was stirred overnight while heating under reflux. After the mixture was allowed to cool, water and methanol were added thereto, and the resulting mixture was stirred, followed by collecting the precipitated solid. The obtained solid was dispersed in toluene at 80° C. for washing, and insoluble matters were filtered off. The filtrate was concentrated to obtain a crude product. The crude product was purified by crystallization from toluene and acetone, and the precipitated solid was collected to obtain 22.1 g of white solid of phenanthrene derivative represented by the formula (I-2) below (yield 75.8%).

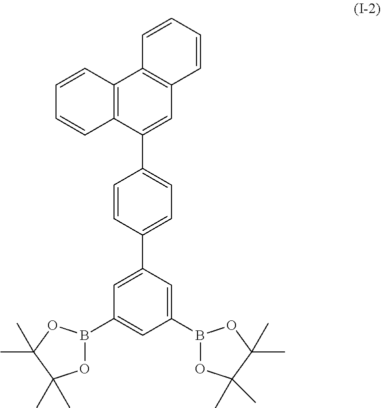

Then, 10.0 g of the phenanthrene derivative represented by the formula (I-2) above, 5.9 g of 2-chloroquinoline, 14.6 g of tripotassium phosphate, 100 ml of 1,4-dioxane, and 30 ml of water were placed in a reaction vessel and mixed. 0.9 g of tris(dibenzylideneacetone)dipalladium(0) and 1.0 g of tricyclohexylphosphine were added thereto, and the mixture was stirred overnight while heating under reflux. After the mixture was allowed to cool, water and methanol were added thereto, and the resulting mixture was stirred, followed by collecting the precipitated solid. The obtained solid was dispersed in monochlorobenzene at 100° C. for washing, and insoluble matters were filtered off. The filtrate was concentrated to obtain a crude product. The crude product was purified by crystallization from monochlorobenzene and acetone, and the precipitated solid was collected to obtain 8.4 g of white powder (yield 83.7%).

The structure of the obtained white powder was identified using NMR.

In ¹H-NMR (CDCl₃), the following signals of 28 hydrogens were detected, and thus it was seen that the obtained powder was Example Compound (1-25).

δ (ppm)=8.97 (1H), 8.83-8.80 (1H), 8.77-8.75 (1H), 8.66-8.65 (2H), 8.33-8.31 (2H), 8.28-8.26 (2H), 8.15-8.13 (2H), 8.06-8.04 (1H), 8.01-7.99 (2H), 7.96-7.94 (1H), 7.90-7.88 (2H), 7.80-7.76 (3H), 7.73-7.56 (8H).

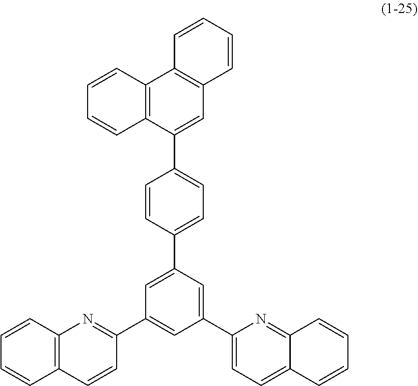 [SMILES: c1ccc2nc(-c3cc(-c4ccc(-c5cc6ccccc6c6ccccc56)cc4)cc(-c4ccc5ccccc5n4)c3)ccc2c1]

### Example 23

### Synthesis of Example Compound (1-26)

Synthesis was performed in the same manner as in Example 22 to obtain 6.5 g of white powder (yield 73.6%), except that 6-chloroquinoline was used instead of 2-chloroquinoline in Example 22.

The structure of the obtained white powder was identified using NMR.

In ¹H-NMR (CDCl₃), the following signals of 28 hydrogens were detected, and thus it was seen that the obtained powder was Example Compound (1-26).

δ (ppm)=8.97-8.96 (2H), 8.82-8.80 (1H), 8.76-8.74 (1H), 8.29-8.26 (4H), 8.20-8.16 (4H), 8.08 (3H), 8.04-8.02 (1H), 7.96-7.92 (3H), 7.77 (1H), 7.73-7.62 (5H), 7.60-7.56 (1H), 7.50-7.46 (2H).

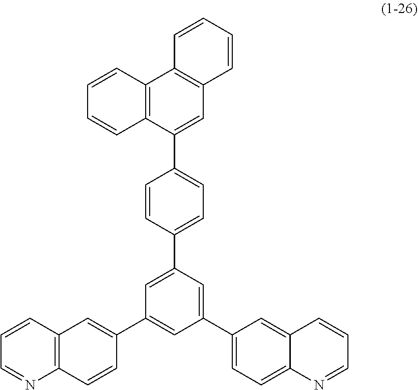 [SMILES: c1cnc2ccc(-c3cc(-c4ccc(-c5cc6ccccc6c6ccccc56)cc4)cc(-c4ccc5ncccc5c4)c3)cc2c1]

### Example 24

The melting point and the glass transition point of each of the compounds obtained in the examples were measured using a high-sensitive differential scanning calorimeter (DSC3100SA manufactured by Bruker AXS K.K.). Table 1 collectively shows the results.

TABLE 1

| | Melting point | Glass transition point |
|---|---|---|
| Compound (1-1) | 267° C. | 101° C. |
| Compound (1-3) | 260° C. | 117° C. |
| Compound (1-49) | 289º C. | 112° C. |
| Compound (1-46) | —° C. | 124° C. |
| Compound (1-47) | —° C. | 125° C. |
| Compound (1-48) | 262° C. | 118° C. |
| Compound (1-17) | —° C. | 137° C. |
| Compound (1-18) | —° C. | 128° C. |
| Compound (1-21) | —° C. | 114° C. |
| Compound (1-22) | —º C. | 105° C. |
| Compound (1-23) | 255° C. | 129° C. |
| Compound (1-24) | —° C. | 122° C. |
| Compound (1-50) | 269° C. | 126° C. |
| Compound (1-33) | 210° C. | 75° C. |
| Compound (1-31) | 217° C. | 102° C. |
| Compound (1-34) | 207° C. | 98° C. |
| Compound (1-35) | —° C. | 134° C. |
| Compound (1-30) | 258º C. | 146° C. |
| Compound (1-36) | 266° C. | 108° C. |
| Compound (1-38) | — ° C. | 120° C. |
| Compound (1-39) | — ° C. | 114° C. |
| Compound (1-25) | 236° C. | 111° C. |
| Compound (1-26) | — ° C. | 118º C. |

It is seen from the results that most of the compounds obtained in the examples had a glass transition point of 100° C. or higher, which indicates that these compounds are stable in the form of a thin film.

### Example 25

A vapor-deposited film with a film thickness of 80 nm was formed on a silicon substrate by using each of the compounds represented by the general formula (1) obtained in the examples, and the refractive index n and the extinction coefficient k at a wavelength of 450 and those at a wavelength of 750 nm were measured using a spectroscopic analyzer (F10-RT-UV manufactured by Filmetrics). For comparison, the measurement was also performed for Comparative Compound (2-1) having the structural formula below and Alq₃ (see Patent Literature 4, for example). All of the compounds of the present invention and the comparative compounds had an extinction coefficient k of 0 in a wavelength range of 450 to 750 nm. Table 2 collectively shows the measurement results of the refractive index n.

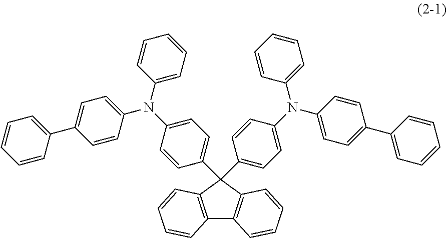 [SMILES: c1ccc(-c2ccc(N(c3ccccc3)c3ccc(C4(c5ccc(N(c6ccccc6)c6ccc(-c7ccccc7)cc6)cc5)c5ccccc5-c5ccccc54)cc3)cc2)cc1]

TABLE 2

| | Refractive index n | Refractive index n |
|---|---|---|
| | (λ 450 nm) | (λ 750 nm) |
| Compound (1-1) | 2.10 | 1.93 |
| Compound (1-3) | 2.03 | 1.88 |
| Compound (1-46) | 2.06 | 1.89 |
| Compound (1-47) | 2.05 | 1.90 |
| Compound (1-48) | 2.01 | 1.87 |
| Compound (1-17) | 2.08 | 1.91 |
| Compound (1-18) | 2.08 | 1.91 |
| Compound (1-21) | 2.03 | 1.87 |
| Compound (1-22) | 1.98 | 1.84 |
| Compound (1-23) | 1.98 | 1.85 |
| Compound (1-24) | 1.98 | 1.84 |
| Compound (1-33) | 1.99 | 1.85 |
| Compound (1-31) | 1.99 | 1.85 |
| Compound (1-34) | 1.99 | 1.83 |
| Compound (1-35) | 1.99 | 1.83 |
| Compound (1-30) | 2.01 | 1.84 |
| Compound (1-36) | 2.03 | 1.87 |
| Compound (1-38) | 2.06 | 1.89 |
| Compound (1-39) | 2.05 | 1.88 |
| Compound (1-25) | 2.04 | 1.90 |
| Compound (1-26) | 2.04 | 1.89 |
| Alq₃ | 1.88 | 1.73 |
| Comparative Compound | 1.84 | 1.77 |
| (2-1) | | |

As shown in Table 2, the refractive indices of the compounds of the present invention were comparable to or higher than those of Alq₃ and Comparative Compound (2-1) in a wavelength range of 450 to 750 nm, which indicates that an improvement in light extraction efficiency can be expected in an organic EL element having a capping layer including any of the compounds of the present invention as a constituent material thereof.

### Example 26

An organic EL element was produced by using a compound of the present invention as a constituent material of the capping layer, and characterized in the atmosphere at normal temperature. Table 3 collectively shows the results of the determination of light emission properties when a DC voltage was applied to the organic EL element.

An organic EL element as shown in FIG. 6 was prepared by providing a glass substrate 1 having a reflecting ITO electrode as a metal anode 2 formed thereon, and then vapor-depositing a hole-injecting layer 3, a hole-transporting layer 4, a light-emitting layer 5, an electron-transporting layer 6, an electron-injecting layer 7, a cathode 8, and a capping layer 9 in this order on the ITO electrode.

Specifically, the organic EL element was prepared in the following manner. A glass substrate 1 on which an ITO film with a thickness of 50 nm, a reflecting film made of silver alloy with a film thickness of 100 nm, and an ITO film with a thickness of 5 nm were formed in this order was ultrasonically cleaned in isopropyl alcohol for 20 minutes, and then dried for 10 minutes on a hot plate heated to 250° C. After that, UV/ozone treatment was performed for 2 minutes. Then, the glass substrate with ITO was set inside a vacuum vapor deposition machine, and the pressure was reduced to 0.001 Pa or less. Subsequently, an electron acceptor (Acceptor-1) having the structural formula below and Compound (3-1) having the structural formula below were vapor-deposited so as to coat the transparent anode 2 through binary vapor deposition at vapor deposition rates such that the ratio of the vapor deposition rate of Acceptor-1 to the vapor deposition rate of Compound (3-1) was 3:97, to thereby form a hole-injecting layer 3 with a thickness of 10 nm.

On the hole-injecting layer 3, a hole-transporting layer 4 (thickness of 140 nm) made of Compound (3-1) having the structural formula below was formed.

Compound (3-2) having the structural formula below and Compound (3-3) having the structural formula below were vapor-deposited on the hole-transporting layer 4 through binary vapor deposition at vapor deposition rates such that the ratio of the vapor deposition rate of (3-2) to the vapor deposition rate of (3-3) was 5:95, to thereby form a light-emitting layer 5 with a thickness of 20 nm.

Compound (3-4) having the structural formula below and Compound (3-5) having the structural formula below were vapor-deposited on the light-emitting layer 5 through binary vapor deposition at vapor deposition rates such that the ratio of the vapor deposition rate of (3-4) to the vapor deposition rate of (3-5) was 50:50, to thereby form an electron-transporting layer 6 with a thickness of 30 nm.

On the electron-transporting layer 6, an electron-injecting layer 7 (thickness: 1 nm) made of lithium fluoride was formed.

On the electron-injecting layer 7, a cathode 8 (thickness: 12 nm) made of magnesium-silver alloy was formed.

Finally, a capping layer 9 (thickness: 60 nm) made of Compound (1-1) of Example 1 was formed.

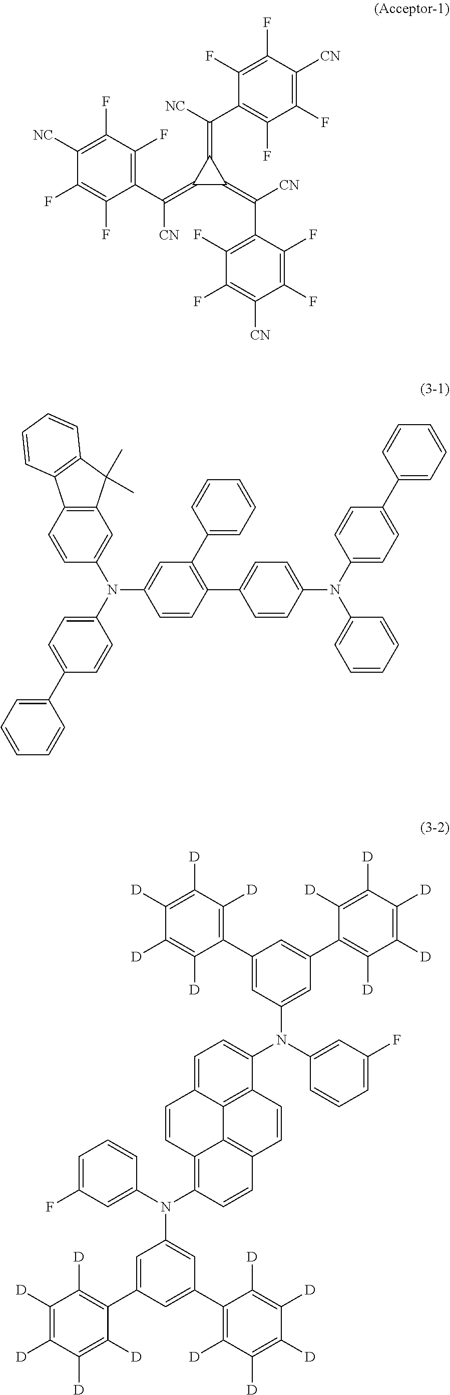 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CC1(C)c2ccccc2-c2ccc(N(c3ccc(-c4ccccc4)cc3)c3ccc(-c4ccc(N(c5ccccc5)c5ccc(-c6ccccc6)cc5)cc4)c(-c4ccccc4)c3)cc21 ; [2H]c1c([2H])c([2H])c(-c2cc(-c3c([2H])c([2H])c([2H])c([2H])c3[2H])cc(N(c3cccc(F)c3)c3ccc4ccc5c(N(c6cccc(F)c6)c6cc(-c7c([2H])c([2H])c([2H])c([2H])c7[2H])cc(-c7c([2H])c([2H])c([2H])c([2H])c7[2H])c6)ccc6ccc3c4c65)c2)c([2H])c1[2H]]

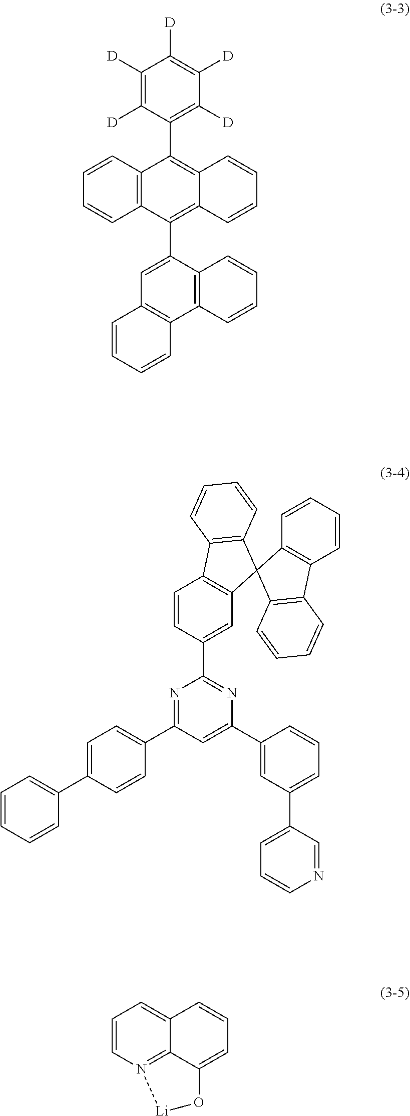

### Example 27

An organic EL element was prepared in the same manner as in Example 26, except that Compound (1-3) of Example 2 was used instead of Compound (1-1) of Example 1 to form a capping laver 9 with a thickness of 60 nm. The organic EL element was characterized in the atmosphere at normal temperature. Table 3 collectively shows the results of the determination of light emission properties when a DC voltage was applied to the organic EL element.

### Example 28

An organic EL element was prepared in the same manner as in Example 26, except that Compound (1-46) of Example 4 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 29

An organic EL element was prepared in the same manner as in Example 26, except that Compound (1-47) of Example 5 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 30

An organic EL element was prepared in the same manner as in Example 26, except that Compound (1-48) of Example 6 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 31

An organic EL element was prepared in the same manner as in Example 26, except that Compound (1-17) of Example 7 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 32

An organic EL element was prepared in the same manner as in Example 26, except that Compound (1-18) of Example 8 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 33

An organic EL element was prepared in the same manner as in Example 26, except that Compound (1-21) of Example 9 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 34

An organic EL element was prepared in the same manner as in Example 26, except that Compound (1-22) of Example 10 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 35

An organic EL element was prepared in the same manner as in Example 26, except that Compound (1-23) of Example 11 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 36

An organic EL element was prepared in the same manner as in Example 26, except that Compound (1-24) of Example 12 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 37

An organic EL element was prepared in the same manner as in Example 26, except that Compound (1-33) of Example 14 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 38

An organic EL element was prepared in the same manner as in Example 26, except that Compound (1-31) of Example 15 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 39

An organic EL element was prepared in the same manner as in Example 26, except that Compound (1-34) of Example 16 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 40

An organic EL element was prepared in the same manner as in Example 26, except that Compound (1-35) of Example 17 was used instead of Compound (1-1) of Example 1 to form a capping layer 9 with a thickness of 60 nm. The organic EL element was characterized in the atmosphere at normal temperature. Table 2 collectively shows the results of the determination of light emission properties when a DC voltage was applied.

### Example 41

An organic EL element was prepared in the same manner as in Example 26, except that Compound (1-30) of Example 18 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 42

An organic EL element was prepared in the same manner as in Example 26, except that Compound (1-36) of Example 19 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 43

An organic EL element was prepared in the same manner as in Example 26, except that Compound (1-38) of Example 20 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 44

An organic EL element was prepared in the same manner as in Example 26, except that Compound (1-39) of Example 21 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 45

An organic EL element was prepared in the same manner as in Example 26, except that Compound (1-25) of Example 22 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Example 46

An organic EL element was prepared in the same manner as in Example 26, except that Compound (1-26) of Example 23 was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Comparative Example 1

For comparison, an organic EL element was prepared in the same manner as in Example 26, except that Alq₃ was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

### Comparative Example 2

For comparison, an organic EL element was prepared in the same manner as in Example 26, except that Compound (2-1) was used instead of Compound (1-1) of Example 1 to form a capping layer 9 with a thickness of 60 nm. The organic EL element was characterized in the atmosphere at normal temperature. Table 3 collectively shows the results of the determination of light emission properties when a DC voltage was applied to the organic EL element.

The element lifespan of each of the organic EL elements prepared in the examples and comparative examples above were measured. Table 2 collectively shows the results. The element lifespan was defined as follows: the organic EL element was driven at a constant current of 10 mA/cm², and the time taken for the luminance to decay to 95% of the initial luminance (100%) was determined and defined as the element lifespan.

TABLE 3

| | | | | Luminance | Power | |
|---|---|---|---|---|---|---|
| | | | Luminance | efficiency | efficiency | Element |
| | | Voltage [V] | [cd/m²] | [cd/A] | [lm/W] | lifespan |
| | Capping layer | (@10 mA/cm²) | (@10 mA/cm²) | (@10 mA/cm²) | (@10 mA/cm²) | (95% decay) |
| Ex. 26 | Compound | 3.61 | 831 | 8.31 | 7.23 | 146 hours |
| | (1-1) | | | | | |
| Ex. 27 | Compound | 3.61 | 798 | 7.98 | 6.95 | 143 hours |
| | (1-3) | | | | | |
| Ex. 28 | Compound | 3.61 | 804 | 8.04 | 7.00 | 157 hours |
| | (1-46) | | | | | |
| Ex. 29 | Compound | 3.63 | 794 | 7.94 | 6.87 | 148 hours |
| | (1-47) | | | | | |
| Ex. 30 | Compound | 3.63 | 817 | 8.17 | 7.06 | 157 hours |
| | (1-48) | | | | | |
| Ex. 31 | Compound | 3.64 | 800 | 8.00 | 6.92 | 158 hours |
| | (1-17) | | | | | |
| Ex. 32 | Compound | 3.64 | 818 | 8.18 | 7.07 | 140 hours |
| | (1-18) | | | | | |
| Ex. 33 | Compound | 3.62 | 788 | 7.88 | 6.85 | 141 hours |
| | (1-21) | | | | | |
| Ex. 34 | Compound | 3.64 | 782 | 7.82 | 6.75 | 163 hours |
| | (1-22) | | | | | |
| Ex. 35 | Compound | 3.65 | 788 | 7.88 | 6.78 | 142 hours |
| | (1-23) | | | | | |
| Ex. 36 | Compound | 3.62 | 773 | 7.72 | 6.71 | 155 hours |
| | (1-24) | | | | | |
| Ex. 37 | Compound | 3.64 | 759 | 7.58 | 6.55 | 165 hours |
| | (1-33) | | | | | |
| Ex. 38 | Compound | 3.64 | 769 | 7.69 | 6.64 | 161 hours |
| | (1-31) | | | | | |
| Ex. 39 | Compound | 3.61 | 770 | 7.70 | 6.70 | 142 hours |
| | (1-34) | | | | | |
| Ex. 40 | Compound | 3.66 | 785 | 7.85 | 6.74 | 166 hours |
| | (1-35) | | | | | |
| Ex. 41 | Compound | 3.64 | 822 | 8.23 | 7.11 | 163 hours |
| | (1-30) | | | | | |
| Ex. 42 | Compound | 3.67 | 812 | 8.12 | 6.96 | 151 hours |
| | (1-36) | | | | | |
| Ex. 43 | Compound | 3.62 | 780 | 7.80 | 6.76 | 157 hours |
| | (1-38) | | | | | |
| Ex. 44 | Compound | 3.65 | 810 | 8.10 | 6.98 | 160 hours |
| | (1-39) | | | | | |
| Ex. 45 | Compound | 3.65 | 827 | 8.28 | 7.12 | 154 hours |
| | (1-25) | | | | | |
| Ex. 46 | Compound | 3.63 | 816 | 8.16 | 7.06 | 164 hours |
| | (1-26) | | | | | |
| Com. | Alq₃ | 3.65 | 714 | 7.14 | 6.15 | 114 hours |
| Ex. 1 | | | | | | |
| Com. | Comparative | 3.65 | 709 | 7.08 | 6.10 | 121 hours |
| Ex. 2 | Compound | | | | | |
| | (2-1) | | | | | |

As shown in Table 3, while the elements of Comparative Examples 1 and 2 and those of Examples 26 to 46 had comparable driving voltages at a current density of 10 mA/cm², all the elements of the examples achieved a significant improvement in the luminance, the luminance efficiency, the power efficiency, and the element lifespan as compared with those of the comparative examples. This fact means that the compound represented by the general formula (1) of the present invention is a material suitable for use in a capping layer, and can increase the refractive index of the capping layer, thereby significantly improving the light extraction efficiency of an organic EL element.

### INDUSTRIAL APPLICABILITY

The compound of the present invention has a high refractive index to significantly improve the light extraction efficiency, and is stable in the form of a thin film, and thus it is a compound suitable for use in an organic EL element. Furthermore, the organic EL element produced by using the compound of the present invention has high efficiency. Furthermore, the compound of the present invention that does not have absorption in blue, green, and red wavelength regions is particularly preferably used to provide a clear and bright image with good color purity. Therefore, the present invention is expected to be applied to applications such as home electric appliances and lighting equipment, for example.

### LIST OF REFERENCE NUMERALS

1 Glass substrate
2 Transparent anode
3 Hole-injecting layer
4 Hole-transporting layer
5 Light-emitting layer
6 Electron-transporting layer
7 Electron-injecting layer
8 Cathode
9 Capping layer

1. A compound represented by a general formula (1) below: 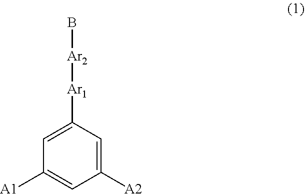
where B represents a substituted or unsubstituted aromatic hydrocarbon group, a substituted or unsubstituted aromatic heterocyclic group, a substituted or unsubstituted fused polycyclic aromatic group, or a substituted or unsubstituted aryloxy group,
Ar₁ and Ar₂ may be the same or different, and represent a substituted or unsubstituted divalent aromatic hydrocarbon group, a substituted or unsubstituted divalent aromatic heterocyclic group, a substituted or unsubstituted divalent fused polycyclic aromatic group, or a single bond, and
A1 and A2 may be the same or different, and represent a monovalent group represented by a general formula (2) below: 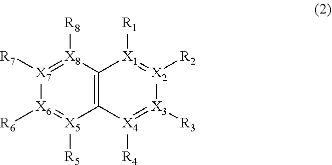
where R₁ to R₈ may be the same or different, and represent a binding site, a hydrogen atom, a deuterium atom, a fluorine atom, a chlorine atom, a cyano group, a nitro group, a linear or branched alkyl group having 1 to 6 carbon atoms and optionally having a substituent, a cycloalkyl group having 5 to 10 carbon atoms and optionally having a substituent, a linear or branched alkenyl group having 2 to 6 carbon atoms and optionally having a substituent, a linear or branched alkyloxy group having 1 to 6 carbon atoms and optionally having a substituent, a cycloalkyloxy group having 5 to 10 carbon atoms and optionally having a substituent, a substituted or unsubstituted aryloxy group, a substituted or unsubstituted aromatic hydrocarbon group, a substituted or unsubstituted aromatic heterocyclic group, or a substituted or unsubstituted fused polycyclic aromatic group,
any one of R₁ to R₈ represents a binding site,
X₁ to X₈ may be the same or different, and represent a nitrogen atom or a carbon atom, and
the number of X₁ to X₈ representing a nitrogen atom is zero to two, and the nitrogen atom does not have a corresponding group of R₁ to R₈ bonded thereto.

2. The compound according to claim 1, wherein A1 and A2 are a monovalent group represented by a general formula (3a), (3b), or (3c) below: 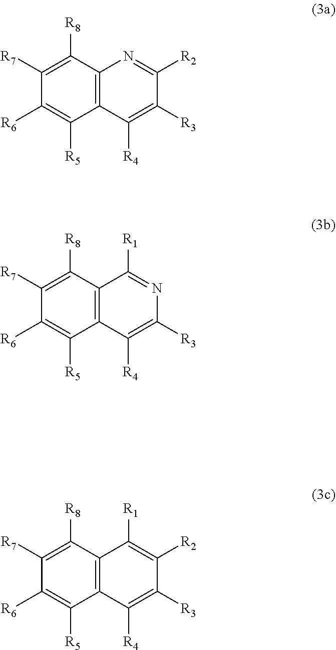
where R₁ to R₈ are the same as defined in the formula (2).

3. The compound according to claim 1, wherein B is a substituted or unsubstituted naphthalenyl group, phenanthrenyl group, dibenzofuranyl group, dibenzothiophenyl group, fluorenyl group, carbazolyl group, benzofuranyl group, or benzothiophenyl group.

4. An organic thin film comprising the compound according to claim 1, having a refractive index of 1.70 or more in a wavelength range of 450 nm to 750 nm.

5. An organic electroluminescent element comprising, at least, an anode, a hole-transporting layer, a light-emitting layer, an electron-transporting layer, a cathode, and a capping layer arranged, in this order,
wherein the capping layer is the organic thin film according to claim 4.

6. An electronic element comprising a pair of electrodes and at least one organic layer interposed therebetween,
wherein the organic layer comprises the compound according to claim 1.

7. An electronic device comprising the electronic element according to claim 6.
